(19) United States
(12) Patent Application Publication
(10) Pub. No.: US 2026/0151261 A1
(43) Pub. Date: Jun. 4, 2026

(54) LUBRICATED FLUID EXCHANGE NEEDLE FOR ENHANCED PERFORMANCE OF REFILLABLE IMPLANTABLE OCULAR DRUG DELIVERY SYSTEM

(71) Applicant: Genentech, Inc., South San Francisco, CA (US)

(72) Inventors: Sara Fermanian, South San Francisco, CA (US); Joshua Horvath, South San Francisco, CA (US); Parul Ohri, South San Francisco, CA (US); John DePalo, South San Francisco, CA (US); Garry Valdez, South San Francisco, CA (US)

(21) Appl. No.: 19/459,350

(22) Filed: Jan. 26, 2026

Related U.S. Application Data
(63) Continuation of application No. PCT/US2024/039560, filed on Jul. 25, 2024.
(60) Provisional application No. 63/516,456, filed on Jul. 28, 2023.
(60) Provisional application No. 63/595,620, filed on Nov. 2, 2023.

(51) Int. Cl. A61F 9/00; A61M 5/32

(52) U.S. Cl. CPC A61F 9/0017; A61M 5/329; A61M 2210/0612

(57) ABSTRACT

An exchange needle device having an elongate needle structure having an elongate tube extending distally from a proximal hub and an outer cannula extending distally from the proximal hub. The outer cannula surrounds at least a proximal end region of the elongate tube forming an annular space between the outer surface of the elongate tube and the inner surface of the outer cannula. The distal end of the outer cannula is located a distance proximal to the distal opening of the elongate tube. A lubricious coating is on the outer surface of the outer cannula and the outer surface of the elongate tube extending distal to the distal end of the outer cannula, the lubricious coating covering at least a portion of the working length of the elongate needle structure. Related devices, systems, and methods of use are provided.

### CROSS-REFERENCE TO PRIORITY DOCUMENTS

This application is a bypass continuation of International Patent Application No. PCT/US 2024/039560, filed Jul. 25, 2024, which claims priority to U.S. Provisional Patent Application No. 63/516,456, filed Jul. 28, 2023; and to U.S. Provisional Patent Application No. 63/595,620, filed Nov. 2, 2023. The disclosures of each of the above-noted applications is incorporated by reference, in its entirety

### BACKGROUND

Diseases that affect vision can be treated with a variety of therapeutic agents, but the delivery of drugs to the eye continues to be challenging. Injections of therapeutic via the eye can be painful, involve some risk of infection, hemorrhage and retinal detachment. Depending on the frequency, intra-ocular injections can be time-consuming for both patient and physician. Consequently, in at least some instances the drug may be administered less often than the prescribed frequency resulting in sub-optimal treatment benefit. Further, bolus intra-ocular injections may not provide the ideal pharmacokinetics and pharmacodynamics. A bolus injection of drug into the vitreous humor of a patient can result in a peak drug concentration several times higher than the desired therapeutic amount and then before the patient is able to get the next injection drop to a drug concentration that is far below therapeutic effectiveness.

Implant devices provide sustained release of a therapeutic drug permitting long-term therapy with fewer injections of the eye. Some implant devices are capable of being refilled while at least partially implanted in the eye allowing for even longer therapy with the same implant device. A needle device can be used to refill the implanted device by penetrating a region of the implanted device. Repeated penetration of the implanted device using a needle device can be problematic for device retention and structural integrity necessary for long-term therapy.

### SUMMARY

In an aspect, provided is an exchange needle device including a proximal hub; and an elongate needle structure having a working length projecting from the proximal hub. The elongate needle structure includes an elongate tube extending distally from the proximal hub. The elongate tube has an outer surface, a proximal end, a distal end, and an inner surface defining a bore extending to a distal opening at the distal end of the elongate tube. An outer cannula extends distally from the proximal hub. The outer cannula has an outer surface, a proximal end, a distal end, and an inner surface defining a bore extending to a distal opening at the distal end of the outer cannula. The outer cannula surrounds at least a proximal end region of the elongate tube forming an annular space between the outer surface of the elongate tube and the inner surface of the outer cannula. The distal end of the outer cannula is located a distance proximal to the distal opening of the elongate tube. The device also includes a lubricious coating on the outer surface of the outer cannula and the outer surface of the elongate tube extending distal to the distal end of the outer cannula. The lubricious coating covers at least a portion of the working length of the elongate needle structure.

The lubricious coating can be a silicone material. The silicone material can be polydimethylsiloxane applied to the portion of the working length using a solvent carrier. The polydimethylsiloxane can be at a concentration of 4-7% w/w in the solvent carrier. The polydimethylsiloxane can have a viscosity in a range of 1,000 cS to about 13,000 cS. The at least a portion of the working length covered by the lubricious coating can include a length from a distal-most end of the needle structure to at least about 2 mm from the distal-most end of the needle structure and no greater than about 5 mm from the distal-most end of the needle structure. The length can be less than an exposed length of the elongate tube and outer cannula relative to the proximal hub. The lubricious coating can cover at least about 50% of the working length of the elongate needle structure starting at a distal-most end of the needle structure up to about 95% of the working length of the elongate needle structure starting at the distal-most end of the needle structure. The proximal hub can have a proximal end that is configured to removably couple to a syringe for delivery of a therapeutic from the syringe through the bore of the elongate tube. The proximal end of the proximal hub can include a Luer connector, a pressure fit connector, or a lock and key mechanism.

The outer cannula can have a region that changes in outer diameter towards the distal end of the outer cannula so that an inner diameter of the outer cannula is smaller at the distal end compared to an inner diameter of the outer cannula near the proximal end of the outer cannula. The inner diameter of the outer cannula at the distal end can contact an outer diameter of the distal end of the elongate tube. The outer cannula can include at least one opening extending through a wall of the outer cannula into the annular space between the outer cannula and the elongate tube. The lubricious coating can extend over the side wall of the outer cannula at a level of the at least one opening and terminates a distance from the proximal hub. The distance can be about 0.25 mm to about 2 mm.

The outer cannula can include a plurality of openings extending through a wall of the outer cannula into the annular space. The plurality of openings can be positioned circumferentially around a longitudinal axis of the outer cannula.

The plurality of openings can be positioned at a plurality of axial locations along a longitudinal axis of the outer cannula. The distal end of the elongate tube can include a sharpened beveled tip. The lubricious coating can be applied by dip-coating the distal end of the elongate tube into a volume of a lubricious material. A positive pressure can be applied through the bore of the elongate tube during insertion of the needle into the volume. The positive pressure through the bore of the elongate tube can be 0.165 psi-0.265 psi. A positive pressure can be applied through the bore of the outer cannula after withdrawal of the needle from the volume. The positive pressure through the bore of the outer cannula can be 15 psi-20 psi. The positive pressure can supply an amount of nitrogen, argon, or air through the bore.

The lubricious material can be partially cross-linked and immobilized by plasma treatment. The lubricious coating on the insertion length can reduce insertion force needed for the insertion length of the exchange needle device to penetrate a septum by at least 25% compared to insertion force for an exchange needle device without the lubricious coating to penetrate a septum. The lubricious coating on the insertion length can reduce insertion force needed for the insertion length of the exchange needle device to penetrate a septum by at least 50% compared to insertion force for an exchange needle device without the lubricious coating to penetrate a septum. The lubricious coating on the insertion length can reduce deformation of a septum during penetration of the septum by the insertion length of the exchange needle device compared to deformation of a septum during penetration of the septum of an exchange needle device without the lubricious coating.

In an interrelated aspect, provided is a method of lubricating a dual lumen exchange needle including dip-coating a length of a dual lumen exchange needle into a volume of a silicone material dissolved into a solvent carrier in a concentration of 4-7% w/w. The dual lumen exchange needle can include an inner tube having an outer surface, a distal end, and a bore extending to a distal opening at the distal end of the inner tube; and an outer cannula having an outer surface, a distal end, and a bore extending to a distal opening at the distal end of the outer cannula The distal end of the outer cannula is located a distance proximal to the distal opening of the inner tube, and the outer cannula surrounds at least a proximal end region of the inner tube forming an annular space between the outer surface of the inner tube and the inner surface of the outer cannula. The method can further include applying a positive pressure through the bore of the inner tube while advancing the needle into the first material; applying a positive pressure through the bore of the outer cannula after withdrawing the needle from the first material; and forming a coating on the length.

The method can further include evaporating the solvent carrier under infrared light. Dip-coating can include a residence time of at least 0 seconds up to about 3 seconds within the material. The method can further include plasma treating the coating to at least partially cross-link and immobilize silicone to the length. The coating can reduce insertion force needed for the exchange needle to penetrate a septum by about 15%-60% compared to insertion force for an exchange needle without the coating. The coating can reduce insertion force needed for the exchange needle to penetrate a septum by about 25%-50% compared to insertion force for an exchange needle without the coating. The coating can reduce deformation of a septum during penetration of the septum compared to deformation of a septum during penetration of the septum without the coating. The length can be at least 2 mm from a distal-most end of the needle and no greater than about 5 mm from the distal-most end of the needle. The inner tube and outer cannula can be coupled on respective proximal end regions to a hub. The length can be less than an exposed length of the inner tube and outer cannula relative to the hub.

The positive pressure through the bore of the inner tube can be 0.165 psi to 0.265 psi. The positive pressure through the bore of the outer cannula can be 15 psi to 20 psi. The silicone material can be polymethylsiloxane and can have a viscosity in a range of 1,000 cS to about 13,000 cS. The coating can cover at least about 50% of a working length of the dual lumen exchange needle starting at a distal-most end of the inner tube up to about 95% of the working length starting at the distal-most end. The length can be less than an exposed length of the inner tube and outer cannula relative to a proximal hub.

In an interrelated aspect, provided is a method of refilling an intraocular drug delivery device with an amount of a therapeutic agent including penetrating a septum of the intraocular drug delivery device with a dual-lumen needle, the drug delivery device at least partially implanted in a vitreous chamber of an eye so the septum is located outside a sclera of the eye and a reservoir of the drug delivery device is located at least partially inside the vitreous chamber. The dual-lumen needle includes an elongate tube having an outer surface, a distal end, and an inner surface defining a bore extending to a distal opening at the distal end of the elongate tube; an outer cannula having an outer surface, a distal end, and an inner surface defining a bore extending to a distal opening at the distal end of the outer cannula. The outer cannula surrounds at least a proximal end region of the elongate tube forming an annular space between the outer surface of the elongate tube and the inner surface of the outer cannula. The distal end of the outer cannula is located a distance proximal to the distal opening of the elongate tube. The device further includes a lubricious coating on the outer surface of the outer cannula and the outer surface of the elongate tube extending distal to the distal end of the outer cannula. The method further includes preventing separation of the septum from the intraocular drug delivery device upon the penetrating due to the lubricious coating.

The lubricious coating can reduce insertion force during the penetrating by at least 25% compared to insertion force for an exchange needle without the lubricious coating. The lubricious coating can reduce insertion force during the penetrating by at least 50% compared to insertion force for an exchange needle without the lubricious coating. The lubricious coating can reduce deformation of the septum during the penetrating compared to deformation of a septum during penetrating without the coating by about 50%.

In some variations, one or more of the following can optionally be included in any feasible combination in the above methods, apparatus, devices, and systems. More details are set further in the accompanying drawings and the description below. Other features and advantages will be apparent from the description and the drawings.

### BRIEF DESCRIPTION OF THE DRAWINGS

These and other aspects will now be described in detail with reference to the following drawings. Generally speaking the figures are not to scale in absolute terms or comparatively but are intended to be illustrative. Also, relative placement of features and elements may be modified for the purpose of illustrative clarity.

FIG. 1 is a cross-sectional, schematic view of a portion of the human eye;

FIG. 2 is a cross-sectional, schematic view of a portion of the human eye having an implementation of a therapeutic device implanted therein;

FIG. 3A is a side view of an implementation of a therapeutic device;

FIG. 3B is a cross-sectional view of the therapeutic device of FIG. 3A taken along line B-B;

FIG. 3C is a side view of the device of FIG. 3A rotated 90 degrees;

FIG. 4 is a schematic of an exchange needle apparatus penetrating a septum of a therapeutic device;

FIG. 5A is a side view of an exchange needle apparatus;

FIG. 5B is a detail view of an elongate structure of the refill needle and hub of FIG. 5A;

FIG. 5C is a cross-sectional view of the elongate structure of FIG. 5C;

FIG. 6 is a cross-sectional view of the connector of the refill needle to couple to a syringe;

FIG. 7 is a detail view of an elongate needle of the exchange needle apparatus having a lubricious coating to reduce the force for septum penetration;

FIGS. 8A-8B illustrate the insertion force (N) to penetrate implants using non-siliconized refill needles compared to siliconized needles;

FIG. 9A shows performance of implants repeatedly penetrated by an uncoated refill needle;

FIG. 9B shows reducing penetration force by siliconizing a refill needle improves septum retention and implant survival upon repeated penetration by the refill needle;

FIG. 9C shows reducing penetration force by siliconizing a refill needle and increasing the septum height and improving geometry of the upper surface relative to the septum improves septum retention and implant survival upon repeated penetration by the refill needle;

FIG. 9D shows reducing penetration force by siliconizing a refill needle without changing the septum height or geometry improves septum retention and implant survival upon repeated penetration by the refill needle;

FIG. 9E shows the combined effect of reducing penetration force by siliconizing a refill needle, increasing the septum height, improving geometry of the upper surface relative to the septum, reducing epoxy and increasing epoxy curing time improves septum retention and implant survival upon repeated penetration by the refill needle;

FIG. 10A shows GCMS data showing significantly higher Trimethyl-1,6-Hexanediamine (TMHMD) residuals within the proximal bodies in which no second epoxy cure was performed;

FIG. 10B shows GCMS data showing significantly lower signals below limit of detection (LOD) for TMHMD residuals within the proximal bodies of a sample in which a second epoxy cure at 150° C. for 3 hours was performed;

FIG. 10C shows GCMS data showing significantly lower signals below LOD for TMHMD residuals within the proximal bodies of a sample in which a second epoxy cure at 125° C. for 3 hours was performed;

FIG. 11A shows overmold separation force in Newtons (N) for an implant without a second epoxy cure compared to an implant having a second epoxy cure at 115-135° C. for 3 hours and an implant having a second epoxy cure at 115-135° C. for 0.5 hours illustrating the second epoxy cure significantly increases the force necessary to separate the overmold from the septum depending on the length of the curing step;

FIG. 11B shows overmold separation force in Newtons (N) for implants with a second epoxy cure at 115° C. and 135° C. for 0.5 hours compared to 3 hours illustrating the effect of time over temperature on the separation force;

FIG. 12 shows a correlation between bond strength of overmolded encasement and septum with punctures-to-failure.

FIG. 13A is a cross-sectional view of a proximal end region of a device illustrating a septum having an upper surface including an untrimmed outer perimeter region;

FIG. 13B is a CT image of a proximal end region of a device having a septum having an upper surface including an untrimmed outer perimeter region in which separation between the untrimmed outer perimeter region of the septum and the encasement occurred due to poor bonding;

FIG. 13C is a CT image of a proximal end region of a device illustrating a septum having an upper surface including an untrimmed outer perimeter region as in FIG. 13A;

FIG. 14A is a cross-sectional view of a proximal end region of the device of FIG. 3B illustrating the fully trimmed upper surface of the septum;

FIG. 14B is a CT image of a proximal end region of a device having a fully trimmed upper surface of the septum bonded to the encasement;

FIG. 14C is a CT image of a proximal end region of a device without an encasement illustrating the fully trimmed upper surface of the septum as in FIG. 14A.

### DETAILED DESCRIPTION

Described herein is a fluid exchange apparatus for refilling implantable devices, systems and methods of use for the delivery of one or more therapeutics for the treatment of diseases.

The implantable therapeutic devices described herein are configured to be repeatedly refilled with therapeutic formulations using a fluid exchange apparatus while positioned within a patient and without explanation of the device. The devices are capable of long-term, minimally-invasive delivery of treatments to the eye. The devices described herein incorporate a septum system that, despite repeated needle penetrations of the septum with a needle, has robust retention relative to the device body. The needle of the fluid exchange apparatus is capable of penetrating the septum of the device using less force and/or deformation, which aids in maintaining the bond between the septum and the body of the implantable device as well as improving user feedback during refill. Additionally, the upper end region of the septum provides a non-planar interface along its entire upper surface that increases the contact area between the septum and an overmold or elastomeric cover or encasement encapsulating the proximal end region of the device. The septum geometry avoids gaps between its perimeter region and the access opening of the device body thereby improving the bond between the septum and the outer encasement. The bond between the septum and the access opening of the device is additionally improved by removing all residual Trimethyl-1,6-hexanediamine (TMHMD) from the septum so that the first bond is substantially free of TMHMD prior to forming a second bond between the encasement and the upper surface of the septum. These and other features of the devices will be described in more detail below.

It should be appreciated that the devices and systems described herein can incorporate any of a variety of features described herein and that elements or features of one implementation of a device and system described herein can be incorporated alternatively or in combination with elements or features of another implementation of a device and system described herein as well as the various implants and features described in U.S. Pat. Nos. 8,399,006; 8,623,395; 9,033,911; 10,500,091; and 9,526,654, the entire disclosures of which are incorporated herein by reference thereto. For the sake of brevity, explicit descriptions of each of those combinations may be omitted although the various combinations are to be considered herein. Additionally, described herein are different methods for implantation and access of the devices. The various implants can be implanted, filled, refilled etc., according to a variety of different methods and using a variety of different devices and systems. Provided are some representative descriptions of how the various devices may be implanted and accessed, however, for the sake of brevity explicit descriptions of each method with respect to each implant or system may be omitted.

It should also be appreciated that the devices and systems described herein can be positioned in many locations of the eye and need not be implanted specifically as shown in the figures or as described herein. The devices and systems described herein can be used to deliver therapeutic agent(s) for an extended period of time to one or more of the following tissues: intraocular, intravascular, intraarticular, intrathecal, pericardial, intraluminal and intraperitoneal. Although specific reference is made below to the delivery of treatments to the eye, it also should be appreciated that medical conditions besides ocular conditions can be treated with the devices and systems described herein. For example, the devices and systems can deliver treatments for inflammation, infection, and cancerous growths. Any number of drug combinations can be delivered using any of the devices and systems described herein.

The materials, compounds, compositions, articles, and methods described herein may be understood more readily by reference to the following detailed description of specific aspects of the disclosed subject matter and the Examples included therein. Before the present materials, compounds, compositions, articles, devices, and methods are disclosed and described, it is to be understood that the aspects described below are not limited to specific methods or specific reagents, as such may vary. It is also to be understood that the terminology used herein is for the purpose of describing particular aspects only and is not intended to be limiting.

### Definitions

Unless defined otherwise, all technical and scientific terms used herein have the same meaning as is commonly understood by one of skill in the art to which the invention(s) belong. All patents, patent applications, published applications and publications, websites and other published materials referred to throughout the entire disclosure herein, unless noted otherwise, are incorporated by reference in their entirety. In the event that there are pluralities of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, relative directional terms such as anterior, posterior, proximal, distal, lateral, medial, sagittal, coronal, transverse, etc. are used throughout this disclosure. Such terminology is for purposes of describing devices and features of the devices and is not intended to be limited. For example, as used herein “proximal” generally means closest to a user implanting a device and farthest from the target location of implantation, while “distal” means farthest from the user implanting a device in a patient and closest to the target location of implantation.

As used herein, a disease or disorder refers to a pathological condition in an organism resulting from, for example, infection or genetic defect, and characterized by identifiable symptoms.

As used herein, treatment means any manner in which the symptoms of a condition, disorder or disease are ameliorated or otherwise beneficially altered. Treatment also encompasses any pharmaceutical use of the devices described and provided herein.

As used herein, amelioration or alleviation of the symptoms of a particular disorder, such as by administration of a particular pharmaceutical composition, refers to any lessening, whether permanent or temporary, lasting or transient that can be attributed to or associated with administration of the composition.

As used herein, an effective amount of a compound for treating a particular disease is an amount that is sufficient to ameliorate, or in some manner reduce the symptoms associated with the disease. Such an amount can be administered as a single dosage or can be administered according to a regimen, whereby it is effective. The amount can cure the disease but, typically, is administered in order to ameliorate the symptoms of the disease. Repeated administration can be required to achieve the desired amelioration of symptoms. Pharmaceutically effective amount, therapeutically effective amount, biologically effective amount and therapeutic amount are used interchangeably herein to refer to an amount of a therapeutic that is sufficient to achieve a desired result, i.e. Therapeutic effect, whether quantitative or qualitative. In particular, a pharmaceutically effective amount, in vivo, is that amount that results in the reduction, delay, or elimination of undesirable effects (such as pathological, clinical, biochemical and the like) in the subject.

As used herein, sustained release encompasses release of effective amounts of an active ingredient of a therapeutic agent for an extended period of time. The sustained release may encompass first order release of the active ingredient, zero order release of the active ingredient, or other kinetics of release such as intermediate to zero order and first order, or combinations thereof. The sustained release may encompass controlled release of the therapeutic agent via passive molecular diffusion driven by a concentration gradient across a porous structure.

As used herein, a subject includes any animal for whom diagnosis, screening, monitoring or treatment is contemplated. Animals include mammals such as primates and domesticated animals. An exemplary primate is human. A patient refers to a subject such as a mammal, primate, human, or livestock subject afflicted with a disease condition or for which a disease condition is to be determined or risk of a disease condition is to be determined.

As used herein, a therapeutic agent referred to with a trade name encompasses one or more of the formulation of the therapeutic agent commercially available under the tradename, the active ingredient of the commercially available formulation, the generic name of the active ingredient, or the molecule comprising the active ingredient. As used herein, therapeutic or therapeutic agents are agents that ameliorate the symptoms of a disease or disorder or ameliorate the disease or disorder. Therapeutic agent, therapeutic compound, therapeutic regimen, or chemotherapeutic include conventional drugs and drug therapies, including vaccines, which are known to those skilled in the art and described elsewhere herein. Therapeutic agents include, but are not limited to, moieties that are capable of controlled, sustained release into the body.

As used herein, a composition refers to any mixture. It can be a solution, a suspension, an emulsion, liquid, powder, a paste, aqueous, non-aqueous or any combination of such ingredients.

As used herein, fluid refers to any composition that can flow. Fluids thus encompass compositions that are in the form of semi-solids, pastes, solutions, aqueous mixtures, gels, lotions, creams and other such compositions.

As used herein, a kit is a packaged combination, optionally, including instructions for use of the combination and/or other reactions and components for such use.

### Eye Anatomy

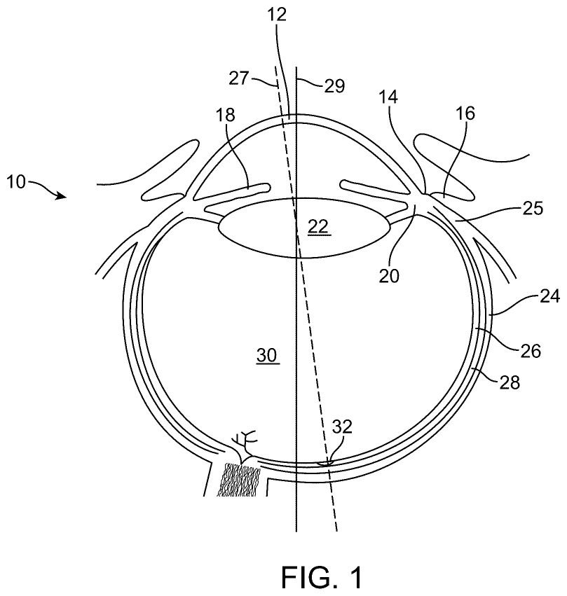

FIG. 1 is a cross-sectional, schematic view of a portion of the human eye 10 showing the anterior chamber, posterior chamber and vitreous of the eye. The eye 10 is generally spherical and is covered on the outside by the sclera 24. The bulk of the eye 10 is filled and supported by the vitreous (referred to herein as vitreous humor or vitreous body) 30, a clear, jelly-like substance disposed between the lens 22 and the retina 26. The retina 26 lines the inside posterior segment of the eye 10 and includes the macula 32. The retina 26 registers the light and sends signals to the brain via the optic nerve. The fovea centralis is the part of the eye located in the center of the macula 32 of the retina 26 and is the region responsible for sharp central vision, for example in order to read or drive. An imaginary line passing from the midpoint of the visual field to the fovea centralis is called the visual axis 27. The hypothetical straight line passing through the centers of curvature of the front and back surfaces of the lens 22 is the optic axis 29.

The elastic lens 22 is located near the front of the eye 10. The lens 22 provides adjustment of focus and is suspended within a capsular bag from the ciliary body 20, which contains the muscles that change the focal length of the lens 22. A volume in front of the lens 22 is divided into two by the iris 18, which controls the aperture of the lens 22 and the amount of light striking the retina 26. The pupil is a hole in the center of the iris 18 through which light entering anteriorly passes. The volume between the iris 18 and the lens 22 is the posterior chamber. The volume between the iris 18 and the cornea 12 is the anterior chamber. Both chambers are filled with a clear liquid known as aqueous humor.

The cornea 12 extends to and connects with the sclera 24 at a location called the limbus 14 of the eye. The conjunctiva 16 of the eye is disposed over the sclera 24 and the Tenon's capsule (not shown) extends between the conjunctiva 16 and the sclera 24. The eye 10 also includes a vascular tissue layer called the choroid 28 that is disposed between a portion of the sclera 24 and the retina 26. The ciliary body 20 is continuous with the base of the iris 18 and is divided anatomically into pars plica and pars plana 25, a posterior flat area approximately 4 mm long.

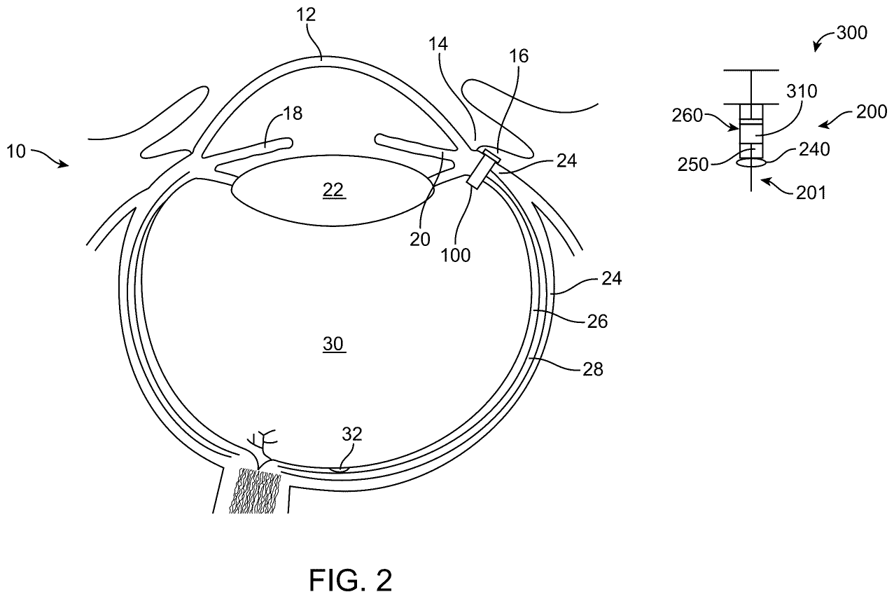

The devices described herein can be positioned in many locations of the eye 10, for example in the pars plana region away from tendon of the superior rectus muscle and one or more of posterior to the tendon, anterior to the tendon, under the tendon, or with nasal or temporal placement of the therapeutic device. As shown in FIG. 2, the devices described herein can be positioned along an axis of insertion A through the sclera 24 in the pars plana region such that the device avoids interfering with the visual field, and in particular, the visual and optic axes 27, 29. The device 100 can be implanted under the conjunctiva 16.

Surgical placement of trans-scleral ocular implants designed to penetrate the globe such that certain regions of the implant occupy supra-scleral, trans-scleral, sub-scleral, and intravitreal aspects of the ocular anatomy in the pars plana region involves a risk of acute vitreous hemorrhage (VH) following surgery. The devices described herein incorporate one or more features that mitigate the risk of vitreous hemorrhage at the time of surgical implantation and improved healing following surgery.

### Treatment Devices

The devices described herein are referred to as drug delivery devices, treatment devices, therapeutic devices, port delivery systems, and the like. It should be appreciated that these terms are used interchangeably herein and are not intended to be limiting to a particular implementation of device over another. The devices and systems described herein can incorporate any of a variety of features described herein and the elements or features of one implementation of a device and system described herein can be incorporated alternatively or in combination with elements or features of another implementation of a device and system described herein as well as the various implants and features described in U.S. Pat. Nos. 8,399,006; 8,623,395; 9,033,911; 10,500,091; and 9,526,654. For the sake of brevity, explicit descriptions of each of those combinations may be omitted although the various combinations are to be considered herein. Additionally, described herein are different methods for implantation and access of the devices. The various implants can be implanted, filled, refilled etc. according to a variety of different methods and using a variety of different devices and systems. Provided are some representative descriptions of how the various devices may be implanted and accessed, however, for the sake of brevity explicit descriptions of each method with respect to each implant or system may be omitted.

The porous structures (also referred to herein as a drug release mechanism, drug release element, release control element, RCE, or frit) as described herein can be used with a number of various different implantable therapeutic devices including one or more of those devices described U.S. Pat. Nos. 8,399,006; 8,623,395; 9,033,911; 10,500,091; and 9,526,654, the entire disclosures of which are incorporated herein by reference thereto.

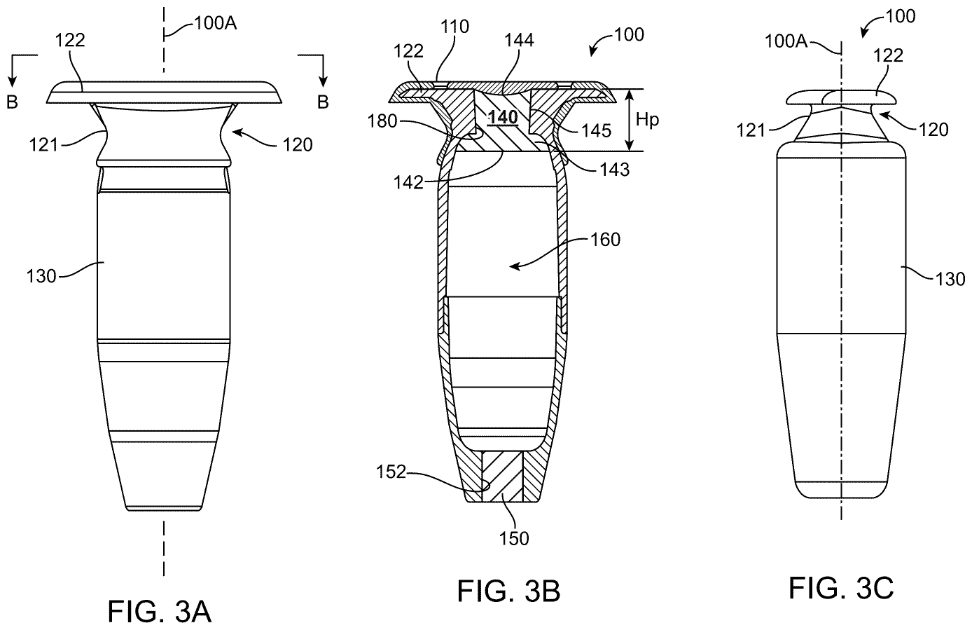

In a first implementation and as shown in FIGS. 3A-3C, the device 100 can include a housing body 130, a penetrable barrier or septum 140 and a porous structure 150. The body 130 can be a rigid, hollow refillable housing for implantation within an interior chamber of the eye, such as the posterior segment of an eye through a penetration in the sclera of the eye. The body 130 need not be rigid and can be pliable so as to expand upon filling, for example, as described in U.S. Pat. No. 11,419,759, which is incorporated by reference herein. The body 130 has a proximal end region and a distal end region. The body 130 has an inner surface that defines, at least in part, a reservoir 160 for holding a therapeutic material or agent(s) (see FIG. 3B). The septum 140 can be positioned within a proximal end region of the body 130 such as within an opening or bore 180 in an access portion of the device that leads into a reservoir 160 of the device. The porous structure 150 can be positioned within another region of the body 130 a distance away from the septum 140 such as within an opening 152 leading out of the reservoir 160 of the device. For example, the porous structure 150 can be positioned near a distal end region of the body 130 opposite the location of the more proximal septum 140. The reservoir 160 can have a volume sized to deliver therapeutic amounts of therapeutic agent to the eye for an extended period of time and the porous structure 150 can be configured to release therapeutic agent contained within the reservoir 160 over the extended period of time. The body 130 can include a proximal retention structure 120 including an extrascleral flange 122 that projects from the proximal end region of the body 130. The extrascleral flange defines an access portion opening or bore 180 that extends from an upper surface of the flange 122 into the reservoir 160 defined by the body 130. The septum 140 can be positioned, at least in part, within the bore 180 such that it forms a seal with the proximal end region of the body 130.

As will be described in more detail below, the devices described herein can also include an elastomeric encasement 110 extending over at least the upper surface of the flange 122 and the upper surface of the septum 140 so as to aid in preventing displacement of the septum 140 relative to the bore 180 upon penetration by needle. The encasement 110 can encapsulate the upper surface and the lower surface of the flange 122 without bonding to the flange 122. The septum 140 can be retained within the device by at least two primary bonds. A first bond is formed between the curved outer surface 145 of the septum 140 and the bore 180 in the proximal body 130 of the device. The first bond between the curved outer surface 145 of the septum 140 and the bore 180 can be formed using an epoxy adhesive that can include an amine-based epoxy curing agent. The encasement 110 can bond only to the upper surface 144 of the septum 140 forming a second bond. The encasement 110 encapsulates the retention structure 120 and bonds to at least the upper surface 144 of the septum 140 of the device. The encasement 110 can be configured to improve the integrity of the septum 140 and its sealing engagement within the bore 180 for repeated injection and long-term implantation. The encasement 110 will be described in more detail below.

Again with respect to FIGS. 3A-3C and as mentioned above, a distal end region of the body 130 can include another opening 152, for example opposite the proximal bore 180 into the reservoir 160, that extends between the inside of the reservoir 160 out of the body 130. The porous structure 150 can be coupled to or positioned, at least in part, within the opening 152. It should be appreciated that the porous structure 150 can be coupled to or positioned within other regions besides the distal end region of the body 130. The porous structure 150 can be affixed within an opening 152 in distal end of body 130, for example with glue or other material(s). Alternatively, or in combination, the distal end of the body 130 can include an inner diameter sized to receive the porous structure 150, and the body 130 can include a stop to position the porous structure 150 at a predetermined location on the distal end so as to define a predetermined size of reservoir 160.

Still with respect to FIGS. 3A-3C, the reservoir 160 within the body 130 of the device 100 can extend along axis 100A between the septum 140 positioned proximally within the bore 180 distally to the location of the porous structure 150. Therapeutic formulations injected into device 100 can be released from the reservoir 160 in accordance with the volume of the reservoir 160 and a release characteristic or release rate index of the porous structure 150. The volume of the reservoir 160 can be sized to deliver therapeutic amounts of a therapeutic agent to the eye for an extended period of time. The volume of the reservoir 160 can be substantially determined by an inner cross-sectional area of the body 130, such as the distance between the proximal, septum 140 and the porous structure 150. The release rate index (RRI) can be used to determine the release of the therapeutic from the device 100. RRI encompasses (PA/FL) where P comprises the porosity, A comprises an effective area, F comprises a curve fit parameter corresponding to an effective length and L comprises a length or thickness of the porous structure 150. Additional details regarding release characteristics of the porous structure 150 that can be used in the various devices described herein can be found, for example, in U.S. Publication No. 2014/0033800, which is incorporated herein by reference in its entirety.

In some implementations, the body 130 can have a dimension such that its length generally exceeds its width or diameter. The body 130 can have a diameter sized within a range, for example, from at least about 0.5 mm to at least about 4 mm, from at least about 1 mm to at least about 3 mm. In some implementations the diameter of the body 130 at its widest point can be about 2.5 mm, for example. The body 130 can have a length sized so as to extend from the conjunctiva 16 to the vitreous 30 along axis 100A to release the therapeutic agent into the vitreous 30. The body 130 can have a length sized within a range, for example, from at least about 2 mm to at least about 14 mm, from at least about 4 mm to at least about 10 mm. In some implementations, the length of the body 130 from the upper-most surface to the distal-most surface can be about 8.5 mm, for example. The flange 122 can remain outside the sclera and have a thickness of about 0.25 mm to about 0.5 mm. The above dimensions are provided as example dimensions and are not intended to be limiting. It should be appreciated that a variety and combination of dimensions are to be considered herein. For example, the body 130 can be configured to expand and thus, the body 130 at its widest point upon filling can be larger than the dimensions described above.

The body 130 and reservoir 160 can each (although not necessarily both) have an elliptical or oval cross-sectional shape, for example. This elongation of the device along one direction can allow for increased drug in the reservoir 160 while at the same time decreasing interference in vision, for example, as the major axis of the ellipse can be aligned substantially with the circumference of the pars plana region 25 of the eye extending substantially around the cornea 12 of the eye, and the minor axis of the ellipse can be aligned radially with the eye so as to decrease interference with vision as the short axis of the ellipse extends toward the optical axis of the eye corresponding to the patient's line of sight through the pupil. Although reference is made to an elliptical or oval cross-section, many cross-sectional sizes and shapes can be used such as circular, square or rectangular with a short dimension extending toward the pupil of the eye and the long dimension extending along the pars plana of the eye.

One or more regions of the body 130 of the devices described herein can be formed of a substantially rigid, biocompatible material. In some implementations, the walls of the body 130 including at least the proximal retention structure 120 down to and including the porous structure 150 are substantially rigid such that the reservoir 160 has a substantially constant volume when the therapeutic agent is released from the device so as to maintain a stable release rate profile, for example when the patient moves. The reservoir 160 can remain substantially rigid and have a substantially constant volume even during injection of the therapeutic agent into the device, for example a device already implanted in the eye.

The devices described herein need not be rigid and can instead include non-rigid walled reservoirs configured to enlarge following implantation such as by filling with treatment solution. The expandable reservoirs may be used with any of the various implementations of a device or system. Further, reference to an expandable reservoir can include a reservoir wall that is pliable and able to be folded, compressed, contracted, etc. into a low profile configuration that is suitable for insertion into the eye in a manner that minimizes the size of penetration. The wall of an expandable reservoir may be pliable or flexible, but need not be stretchy or elastomeric in order to enlarge in size to hold the treatment solution. The expandable reservoir can include a reservoir wall that tents, unfolds, expands, stretches, or otherwise enlarges the overall cross-sectional size of the reservoir compared to the low profile configuration suitable for insertion. It should be appreciated that the terms unfold, expand, enlarge, and other terms used to refer to this shape change of the reservoirs described herein may be used interchangeably.

One or more regions of the body 130, one or more regions of the retention structure 120 as well as other portions of the devices described herein, alone or in combination, can be formed of one or more of many biocompatible materials including, but not limited to materials such as acrylates, polymethylmethacrylate, siloxanes, metals, titanium stainless steel, polycarbonate, polyetheretherketone (PEEK), polyethylene, polyethylene terephthalate (PET), polyimide, polyamide-imide, polypropylene, polysulfone, polyurethane, polyvinylidene fluoride, polyphenylene polyphenylsulfone or PTFE, and others. Alternatively or in combination, one or more portions of the devices described herein, such as the body 130, can be formed at least in part from an optically transmissive material such that the body 130 can be translucent or transparent, such that when the device 100 is loaded with therapeutic agent the reservoir 160 can be visualized outside the patient prior to implantation, for example when injected with a formulation of therapeutic agent prior to implantation in the physician's office. The encasement 110 can also be a transparent or translucent material for visualization of the septum 140 of the device. This visualization of the reservoir 160 can be helpful to ensure that the reservoir 160 is properly filled with therapeutic agent by the treating physician or assistant prior to implantation. For example, transparency can enable visualization, for example, using an indirect ophthalmoscope, of the contents of the reservoir 160 of an implanted device allowing one to confirm that no air is trapped in the device and/or verify the clarity of the device contents. A cloudy appearance, for example, may indicate that some degree of contamination, microbial or otherwise, has occurred. The biocompatible, optically transmissive materials can include one or more of acrylate, polysulfone, polyacrylate, methlymethacrylate, polymethylmethacrylate (PMMA), polycarbonate, glass or siloxane.

The porous structure 150 can include one or more of a release control element, a release control mechanism, permeable membrane, a semi-permeable membrane, a material having at least one hole disposed therein, channels formed in a rigid material, straight channels, nano-channels, nano-channels etched in a rigid material, laser drilled holes, laser etched nano-channels, a capillary channel, a plurality of capillary channels, one or more tortuous channels, sintered material, sintered rigid material, sintered glass, sintered ceramic, sintered metal, sintered titanium, tortuous micro-channels, sintered nano-particles, an open cell foam or a hydrogel such as an open cell hydrogel. Porous structures considered herein are described in U.S. Pat. Nos. 8,399,006; 8,623,395; 9,033,911; and US Publication No. 2014/0033800, filed Nov. 10, 2011, the entire disclosures of which are incorporated herein by reference thereto.

Again with respect to FIGS. 3A-3C and as mentioned above, the retention structure 120 can protrude outward from the proximal end region of the body 130. At least a portion of the underside of the retention structure 120 can contact the sclera 24 and at least a portion of the upper side of the retention structure 120 can contact the conjunctiva 16. In some implementations, the retention structure 120 can be configured to contact the sclera 24 such that the retention structure 120 is at least partially embedded within the thickness of the sclera 24 and does not necessarily sit on an upper surface of the sclera or the conjunctiva. The retention structure 120 can have a thickness between the underside and the upper side that is between about 0.25 mm to about 0.5 mm.

The retention structure 120 can include a narrowed portion 121 and a wider, extrascleral flange 122 extending proximally from the narrowed portion 121. The narrowed portion 121 can have a cross-section sized to fit in an elongate incision or a puncture through the pars plana region 25 without causing gaping of the tissue near either end of the incision. For example, an incision can be made with a device having a straight, flat blade, for example a 3.2 mm blade. Penetrating the sclera with such a blade can result in exposed scleral tissue that may need to be sealed (e.g., 6.4 mm or 2×3.2 mm). A cross-sectional region of an implant positioned within the cut region of the sclera, for example having a perimeter of 6.4 mm and a diameter of about 2 mm, could open the wound such that there would be relatively large voids on either side of the device, for example about 2.2 mm between either side of the device and the farthest aspects of the exposed sclera. These voids can result in cut portions of the sclera remaining exposed and unsealed. The geometry of the narrowed portion 121 of the devices described herein can be designed to minimize the length of cut scleral tissue that remains exposed and/or unsealed.

The narrowed portion 121 can have a first cross-sectional distance across, or first dimensional width, and a second cross-sectional distance across, or second dimensional width, in which the first cross-sectional distance across is greater than the second cross-sectional distance across providing the narrowed portion 121 with an elongate cross-sectional profile. The elongate cross section of the narrowed portion 121 can be sized in many ways to fit the incision. The elongate cross section can have a first dimension longer than a second dimension and can have one or more of many shapes such as dilated slit, dilated slot, lentoid, oval, ovoid, or elliptical. It should also be appreciated that the narrowed portion 121 can have other cross-sectional shapes, for example, a circular shape, if desired. The dilated slit shape and dilated slot shape can correspond to the shape assumed by the scleral tissue when cut and dilated. The lentoid shape can correspond to a biconvex lens shape. The elongate cross-section of the narrowed portion 121 can include a first curve along a first axis and a second curve along a second axis that is different than the first curve. The narrowed portion 121 can be sized and configured to receive the sclera 24 upon implantation in the eye 10 when the flange 122 is positioned between the sclera 24 and the conjunctiva 16 and the distal end of the body 130 extends into the vitreous 30.

Flange 122 of the retention structure 120 can include a first distance across and a second distance across. The first distance across can be greater than the second distance across (see FIGS. 3B and 3C, for example). The first distance across can result in the flange 122 having a diameter greater than a largest diameter of the body 130 (see e.g., FIG. 3B) whereas the second distance across can result in the flange 122 having a diameter equal to or less than a largest diameter of the body 130 (see e.g., FIG. 3C). The flange 122 can have a variety of shapes, such as rectangular, square, oval, elliptical, circular, teardrop, polygonal or other shape. The flange 122 can be formed as a smooth protrusion configured for placement along a portion of the sclera 24. In some implementations, the flange 122 is positioned under the conjunctiva 16, such that the conjunctiva 16 covers and protects the device 100. Coverage of the flange 122 by the conjunctiva 16 can also aid in preventing infection by providing a barrier. The flange 122 can be formed from a translucent material such that the physician can visualize tissue under the flange 122 to assess the patient and to decrease appearance of the device 100 when implanted.

As mentioned above, the septum 140 can be positioned, at least in part, within bore 180 sealing the reservoir 160 on a proximal end region of the device 100 (see FIG. 3B). The septum 140 can be a septum configured to receive and be repeatedly penetrated by a sharp object such as a needle of the fluid exchange apparatus for injection of the therapeutic agent into the reservoir 160. The septum 140 can be configured to re-seal when the sharp object is removed. The septum 140 can be a pre-molded soft, high strength material. The septum 140 need not be pre-molded to the exact shape and size of the bore 180 within which it is to be positioned. Preferably, the septum 140 is slightly oversized relative to the bore 180 such that upon positioning of the septum 140 within the body 130, the inner surfaces of the bore 180 apply an amount of radial compression on the pre-molded septum 140. The body 130 defining the bore 180 can be formed of a higher durometer material compared to the material of the septum 140 such that the smaller dimension of the higher durometer body 130 applies compression and provides additional septum seal performance, such as by encouraging re-sealing of the septum 140 after penetration and removal of a needle track during refilling of the reservoir. In some implementations, the septum 140 can be formed from one or more elastic materials such as siloxane, rubber, or another liquid injection molding silicone elastomer such as NUSIL MED-4810 (NuSil Silicone Technology, Carpinteria, CA), or other elastomer such as polyurethane elastomers (thermoplastic urethanes) or polyurethane/siloxane copolymers. In some implementations, the septum 140 can include an opaque material and/or a colored material such that it can be visualized by the treating physician.

As described above and as best shown in FIGS. 3A-3C, the septum 140 can be positioned within a proximal end region of the body 130 at least in part within the bore 180 of the access portion. As such, the overall shape of the external surface of the septum 140 can correspond generally to the shape of the surface(s) near the bore 180 against which the septum 140 contacts to mate and seal. It should be appreciated that the points of contact between the septum 140 and the body 130 can vary. The septum 140 can make contact, for example sealing contact, with at least one or more surfaces or regions of the upper end of the reservoir chamber, the body 130, the retention structure 120, the narrowed portion 121, the flange 122, the bore 180, and/or a combination thereof.

The septum 140 can have an upper surface 144, a middle region 145 formed by a curved outer surface, and a lower surface 142 on a distal end region 143 of the septum 140 (see, e.g., FIG. 3B). The upper surface 144 can be sized to reside within and mate with at least a portion of the flange 122, such as an upper end of the bore 180. The upper surface 144 of the septum 140 can be available through the access region opening or bore 180 of the device. The middle region 145 can be sized to reside within and mate with inner surfaces of the narrowed portion 121 of the retention structure 120 defining the bore 180. The middle region 145 can be a reduced diameter region or form a “waist” in the septum 140.

In some implementations, the distal end region 143 can have a diameter that is the same as or greater than the narrowed portion 121 of the retention structure 120. For example, the distal end region 143 of the septum can be formed by one or more tabs, a flared skirt, flange, rib or other feature of enlarged diameter compared to the middle region 145 and/or upper surface 144 sized to reside within and mate with at least a portion of the retention structure 120 located distal to the narrowed portion 121 and/or an upper region of the reservoir 160 such as with an inner wall of the body 130. The distal end region 143 of the septum 140 is configured to contact an inner wall surface near the upper end of the reservoir 160 that surrounds the bore 180 of the access portion (see FIG. 3B). The features of the distal end region 143 having an enlarged diameter compared to the middle or upper regions of the septum 140, such as the one or more tabs, flanges, or flared skirt, can also aid in retaining the septum 140 within the bore 180 during manufacturing. The cross-sectional diameter of the septum 140 distal to the middle region 145 in at least a first direction can be equal to, more or less than the cross-sectional diameter of the upper surface 144.

Repeated injection as well as long-term implantation of the device 100 can affect the integrity of the septum 140. For example, repeated injection through the septum 140 can at least partially damage the device and negatively affect the seal between the inner surfaces of the body 130 and the outer surfaces of the septum 140. Further, over time after implantation the septum 140 can loosen relative to the body 130. Described herein are features to improve the integrity of the septum 140, its sealing engagement with the bore 180 of the body 130 and/or retention structure 120, and the effectiveness of the access region for repeated injection and long-term implantation of the re-fillable devices described herein.

As mentioned above, the devices described herein can be coupled to a encasement 110 configured to improve the integrity of the septum 140 and its sealing engagement with the bore 180 for repeated injection and long-term implantation. This provides a benefit to a device intended to be implanted long-term and re-filled while implanted, such as those described herein. The encasement 110 is coupled to at least a proximal end region of the device so as to encapsulate the retention structure 120 and bond to the upper surface of the septum 140. For example, the encasement 110 can couple to at least a portion of a proximal end region of the device 100, including one or more combinations of the upper surface of the septum 140 positioned within the opening of the access portion bore 180, an upper surface of the proximal retention structure 120 including the flange 122, a lower surface of the proximal retention structure 120 including the flange 122, the narrowed portion 121 of the retention structure 120, and at least a portion of an outer surface of the body 130 near the proximal end region.

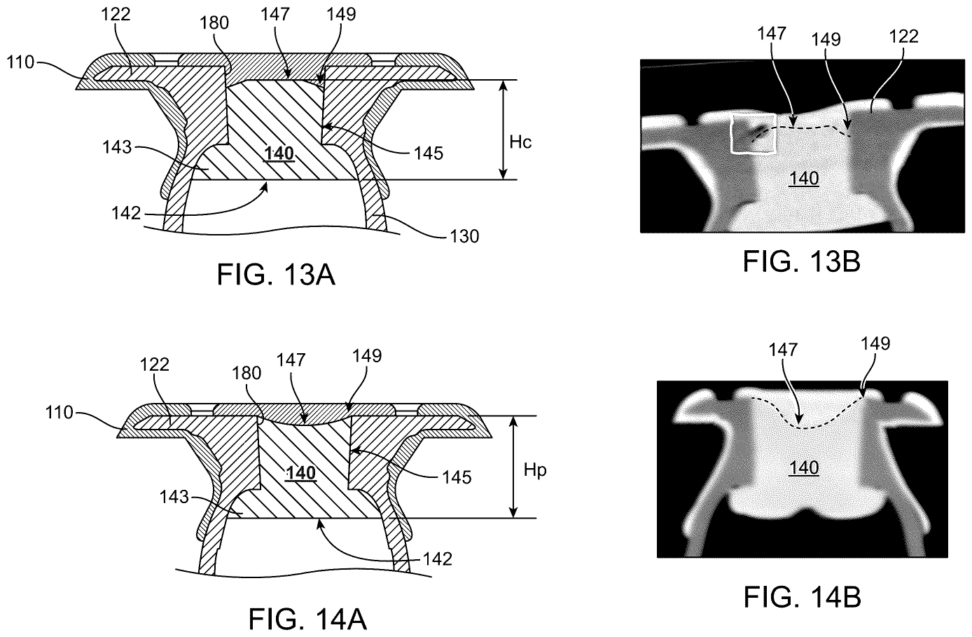

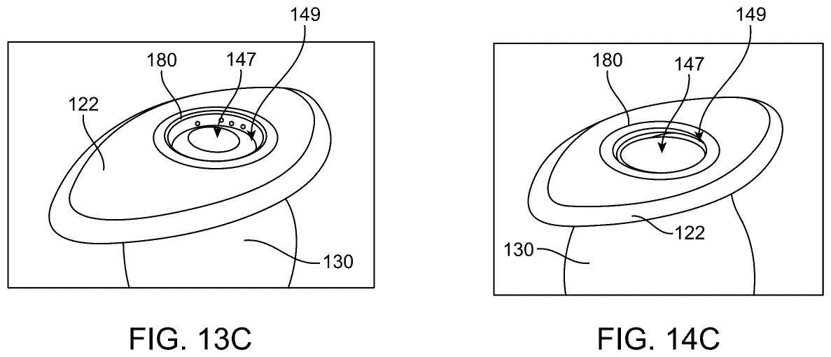

The encasement 110 and the proximal retention structure 120 (or any other region coupled to the encasement 110 such as the flange 122), can have corresponding shape profiles. The thickness of the over-molded encasement 110 can vary from approximately 0.007″ (0.180 mm) to approximately 0.025″ (0.640 mm). In some implementations, the septum 140 had a concave or conical shaped upper surface in which a central portion of the septum 140 lies below the outer perimeter of the septum 140 forming a depression within the central portion. The thickness of the over-molded encasement 110 in the region of the depression can exceed 0.025″ without significantly increasing the effective thickness of the flange 122 or shape profile of the extrascleral portion of the device. The encasement 110 can extend beyond the outer diameter of the flange 122 (see FIGS. 13A and 14A). The encasement 110 can also extend upward from the upper surface of the flange 122 and provide a slightly thicker and slightly higher profile to the access portion under the conjunctiva. During injection of the therapeutic agent into the reservoir 160 using the fluid exchange apparatus, the needle can extend through the encasement 110 as it penetrates the septum 140. The encasement 110, like the septum 140, can be configured to re-seal when the needle or other sharp object is withdrawn.

The proximal retention structure 120 can include one or more through-holes, apertures, indentations or other features. The flange 122 can include one or more apertures extending therethrough. Upon application of the encasement 110, the apertures create mechanical struts of the over-molded encasement material that extend through one or more regions of the flange 122. The mechanical struts of over-molded encasement material provide some anchoring support as well as facilitate good filling of the over-mold. The apertures can also allow for a thin, uniform layer of over-mold material to form on the underside of the flange 122 or other another region of the retention structure during over-molding. It should be appreciated, however, that mechanical struts of the over-molded material can be formed by over-molded material extending only partially through apertures in the flange 122. Further, instead of apertures, the flange 122 can include only partial-thickness holes or indentations in the flange 122. The indentations can be on an external surface of the flange 122 such as in the upper and/or lower surfaces of the flange 122. The external surfaces of the flange 122 can also be textured such that the over-molded material of the encasement 110 can penetrate and fill additional indented regions of the flange 122 to provide a better coupling between the flange 122 and the material of the encasement 110.

The proximal end region of the body 130 of the therapeutic device 100 can be machined from a piece of material, or injection molded, so as to form the retention structure 120, flange 122 and/or the narrowed portion 121. As described above, the septum 140 can be pre-molded and the encasement 110 can be over-molded. Alternatively, the encasement 110 can be pre-molded and bonded to the pre-molded septum 140. The septum 140 and encasement 110 can be the same material and over-molded around the flange 122 using a single step injection molding process. Alternatively, the septum 140 or encasement 110 can be two different materials and over-molded around the flange and cured in two independent steps.

The septum 140 preferably bonds along its waist, or the curved outer surface of the middle region 145 connecting the upper surface 144 to the lower surface 142 of the septum 140, to the inner surfaces of the bore 180 of the body 130. The septum 140 bonds to the encasement 110 along its upper surface. The encasement 110 can bond only to the septum 140 although it may encapsulate the flange 122 of the body 130. The proximal end region of the body 130 can be any of a variety of materials such as polysulfone. The septum 140 positioned within the proximal end region of the body 130 can be pre-molded, soft, high-strength material such as a liquid injection molding silicone elastomer such as MED-4810 (NuSil Silicone Technology, Carpinteria, CA). The encasement 110 can be an over-molded, high durometer material such as a translucent, liquid silicone rubber like MED-4880 or MED-4860 (NuSil Silicone Technology, Carpinteria, CA).

FIGS. 13A-13B show an implementation of a device in which the bore 180, the septum 140 and the retention structure 120 are over-molded by the encasement 110. The encasement 110 encapsulates the upper surface of the proximal retention structure 120 and the lower surface of the proximal retention structure 120. The upper surface of the septum 140 is accessible from the proximal end region of the device to as to bond to the encasement 110. As discussed elsewhere herein, the septum 140 can be a pre-molded element having a first shape that is installed within the bore 180 at the proximal end region of the device and cut to a second shape following positioning within the bore 180. Trimming the septum 140 to a second shape exposes an interior of the septum 140 maximizing the surface area available for improved bonding. The curved outer surface of the middle region 145 of the septum 140 engages with the inner surface of the body 130 forming the bore 180. In the implementation shown in FIGS. 13A-13C, the septum 140 installed within the bore 180 is trimmed only at the central region 147 and is left untrimmed at the outer perimeter region 149. The trimmed central region 147 can be substantially planar and the untrimmed outer perimeter region 149 lies below the plane of the central region 147. The entire septum 140, including the planar central region 147 and the beveled outer perimeter region 149 lying below the plane of the central region 147, lies below the plane of the upper surface of the flange 122 of the retention structure 120 such that the septum 140 is countersunk relative to the flange 122. The interface between the encasement 110 and the septum 140 is less than a full diameter of the septum 140 and relies upon the substantially planar contact area with the central region 147. The septum 140 can be placed under tension prior to trimming to achieve the second shape.

The septum 140 positioned into a proximal region of the device 100 can be maintained in the bore 180 in an adhesion-free manner and rely on the mating features between the external surface of the septum 140 with the corresponding surfaces of the bore 180 against which the septum 140 abuts and seals. Preferably, the septum 140 is adhered within the bore 180 of the retention structure 120, such as by a two-part epoxy. Two-part epoxies include a Part A resin and a Part B hardener. Amine-based curing agents may be used in the epoxy hardener. Platinum catalysts can be susceptible to poisoning from amine-based epoxy curing agents. Thus, the epoxy fixation between the septum 140 and the body 130 can impair the bonding between the septum 140 and the encasement 110.

Again with respect to FIGS. 13A-13C, the countersunk septum 140 can create gaps near where the beveled outer perimeter region 149 lying below the plane of the central region 147 meets the inner surfaces of the bore 180. For example, the maximum height Hc between the lower surface 142 of the septum 140 and the planar upper surface of the central region 147 is greater than the height Hp of the septum 140 between the lower surface 142 and the outermost edge of the perimeter region 149. While the planar upper surface of the central region 147 may extend clear to the upper surface of the flange 122, the shorter height Hp of the outer perimeter region 149 forms a sunken region of the septum 140 within the bore 180. This sunken region creates a gap or an upper region of the bore 180 that is not bonded to the curved outer surface 145 of the septum 140. This gap can collect and trap epoxy used to bond the septum 140 to the body 130. The excess epoxy in this region can impair or prevent bonding between the upper surface of the septum 140 and the elastomeric encasement 110.

FIGS. 14A-14C show another implementation of a device in which the bore 180, the septum 140 and the retention structure 120 are over-molded by the encasement 110. The septum 140 is pre-molded to a first shape and cut to a second shape once the septum 140 is positioned within the bore 180. Trimming the septum 140 to the second shape exposes an interior of the septum 140. The exposed interior spans across the full upper surface of the septum 140. The septum 140 installed within the bore 180 at the proximal end region of the device is trimmed to provide a septum/encasement interface that spans the full diameter of the septum 140 thereby eliminating any gaps or sunken region at an upper region of the bore 180 that might trap epoxy between the encasement 110 and the septum 140. The curved outer surface 145 of the septum 140 in FIGS. 14A-14B engages with the inner surface forming the bore 180 up to or near a level of the upper surface of the flange 122 minimizing or eliminating the sunken region that can trap epoxy.

The entirety of the upper surface of the septum 140 is trimmed including the trimmed central region 147 and the trimmed outer perimeter region 149. The trimmed central region 147 is concave or conical in shape prior to installation of the encasement 110 forming a depression in the upper surface of the septum 140. The concave or conical shape increases the surface area available for the encasement 110 and the septum 140 to bond compared to that provided by the planar central region 147 in the embodiment shown in FIGS. 13A-13B. The material of the encasement 110 can fill the depression in the upper surface of the septum 140. A portion of the outer perimeter region 149 forming a proximal-most surface of the septum 140 lies above a plane of the central region 147 so that the central region 147 of the upper surface of the septum lies below the perimeter region 149. While the central region 147 lies below the plane of the upper surface of the flange 122 of the retention structure 120, the outer perimeter region 149 of the upper surface of the septum lies flush or substantially flush with the plane of the upper surface of the flange 122. The central region 147 can be countersunk, but the outer perimeter region 149 is not counter sunk relative to the flange 122. In this implementation, the interface between the encasement 110 and the septum 140 is across the full diameter of the septum 140 and includes an increased contact area of both the outer perimeter region 149 and the central region 147. The contact area at the interface is non-planar (e.g., concave depression). The outer perimeter region 149 lying substantially flush with the plane of the upper surface of the flange 122 minimizes or eliminates any gaps between the outer perimeter region 149 and the inner surfaces of the bore 180. The maximum height Hp of the septum 140 between the lower surface of the septum 140 to the upper surface of the outer perimeter region 149 can be greater than the height Hc of the septum 140 between the lower surface and the upper surface of the central region 147. The maximum height Hp can be greater than about 1.05 mm and less than about 1.35 mm. The epoxy bond between the curved outer surface of the septum and the bore 180 covers the entire surface of the bore 180 up to or near a level of the upper surface of the flange 122 compared to that of FIGS. 13A-13B in which the epoxy bond covers only a partial portion of the bore 180. Additionally, no gaps exists at the outermost edge of the perimeter region 149 that can collect and trap epoxy, thereby improving the bond between the material of the septum 140 and the material of the encasement 110. The larger trimmed surface of the septum 140 for bonding to the encasement 110 improves the bond between the material of the septum 140 and the material of the encasement 110.

FIG. 13B is a CT image of a proximal end region of a device having a septum 140 with an untrimmed outer perimeter region 149 that is lying below the plane of the upper surface of the flange 122 creating a sunken region or a gap within which epoxy can collect. The image illustrates separation (see square drawn in FIG. 13B) that occurred due to poor bonding between the outer perimeter region 149 of the septum 140 and the encasement 110. The encasement 110 has lifted away from the beveled outer perimeter region 149 lying below the plane of the flange 122 and remains bonded only at the central region 147. FIG. 14B is a CT image of a proximal end region of a device having a fully trimmed upper surface of the septum 140 from the outer perimeter region 149 to the central region 147. The outer perimeter region 149 lies substantially flush with the plane of the upper surface of the flange 122 and the central region 147 is countersunk into a concave or conical shape below the plane of the flange 122. The encasement 110 is fully bonded across the entire span of the septum 140 available through the bore 180 of the device.

FIG. 13C is another CT image of the proximal end region of a device without the encasement 110 present. The septum 140 is visible within the bore 180 of the device. The central region 147 is trimmed into a concave or conical shape as opposed to a planar surface. However, the septum 140 includes an outer perimeter region 149 that is untrimmed and lies below the plane of the upper surface of the flange 122. FIG. 14C is a CT image of the proximal end region of a device also without a encasement 110 present. The septum 140 is visible within the bore 180 and shows a fully concave shape over the central region 147 such that the outer perimeter region 149 forms an outermost edge of the septum 140 that lies substantially even with the upper surface of the flange 122 so that any gaps in this outer perimeter region 149 are eliminated.

A two-part epoxy can be used to form the first bond between the septum 140 and the proximal body 130 of the implant device. The geometry of the septum 140 relative to the bore 180 improves retention of the septum 140 over time and upon repeated penetrations by mitigating inadvertent exposure of the silicone to the epoxy. The septum 140 can be a silicone material and the proximal body 130 defining the bore 180 can be a material such as polysulfone. Residual materials from the hardener or Part B of the epoxy (e.g., Trimethyl-1,6-hexanediamine, TMHMD) can interfere with silicone polymerization needed to bond the silicone material of the septum 140 to the silicone material of the encasement 110. TMHMD residue can persist in septum/proximal body subassemblies prior to overmolding with the encasement 110, even if no traces of TMHMD are found in the finished implant. As will be described in more detail below, in addition to the geometry of the septum 140 relative to the bore 180 discussed above, additional curing steps of the septum/proximal body subassemblies (i.e., the septum 140 assembled and adhered within bore 180 of the proximal body 130 of the implant device 100) improves the bond strength between the septum 140 and the encasement 110 by vaporizing and removing residual TMHMD from the subassemblies prior to assembly and bonding with the encasement 110.

The curved outer surface of the septum 140 forms a first bond with the bore 180 of the body 130 using an epoxy adhesive, which can include an amine-based epoxy curing agent. The first bond is substantially free of TMHMD prior to forming the second bond between the encasement 110 and the upper surface of the septum 140. The first bond between the curved outer surface of the septum 140 and the bore 180 if the body 130 is cured in at least two stages prior to forming the second bond between the encasement 110 and the upper surface of the septum 140. The first stage of curing the septum/proximal body subassembly includes heating the first bond between the septum 140 adhered within the bore 180 of the body 130 to about 67° C. for at least 1 hour up to about 3 hours. The second stage of curing includes heating the first bond to at least 115-135° C. for more than 30 minutes and less than about 4 hours, preferably about 3 hours. The bond strength between the septum 140 and the encasement 110 is greatly improved by the second curing stage of the septum 140 to the body 130 because the second curing stage eliminates the TMHMD residue in the epoxy that impairs the bond between the septum 140 and the encasement 110.

The overall robustness of the device is increased by increasing septum height relative to the bore within which it is bonded and increasing the trimmed septum surface area available for bonding to the encasement to eliminate gaps at the outer perimeter region 149 of the septum 140 and minimizing the epoxy contamination. The overall robustness of the device is also increased by reducing residual TMHMD from the septum/proximal body subassembly prior to overmolding with the encasement 110 by adding a second stage of epoxy curing that is performed at a temperature of at least 115° C. and for greater than 0.5 hour and less than about 4 hours.

It should be appreciated the residual TMHMD can be removed in other ways besides oven heating for curing, such as using solvent cleaners or reactive gas. Reducing the amount of epoxy dispensed to bond the septum to the proximal body can aid in reducing the residual TMHMD found in the constructs. For example, the epoxy dispensed within the bore 180 (or dispensed onto the curved outer surface of the septum 140) can be less than about 0.30 uL, less than about 0.25 uL, less than about 0.20 uL, or less than about 0.15 uL down to about 0.05 uL. Reducing the epoxy dispensed can minimize excess epoxy and its adverse impact on overmold/septum bonding.

Certain surface treatments can also be used during manufacturing of the devices described herein to enhance bonding between various components, including for example but not limited to, bonding primer agents such as NUSIL MED 161 or other surface activation techniques such as plasma treatment of the surfaces to be bonded. For example, the bore 180 within which the septum 140 is positioned can undergo plasma treatment. For example, the bore 180 within which the septum 140 is positioned can undergo plasma treatment. Air plasma treatment of about 3 seconds up to about 5 seconds can be used on the septum 140 while rotating the septum for the treatment duration.

### Fluid Exchange Needle Device

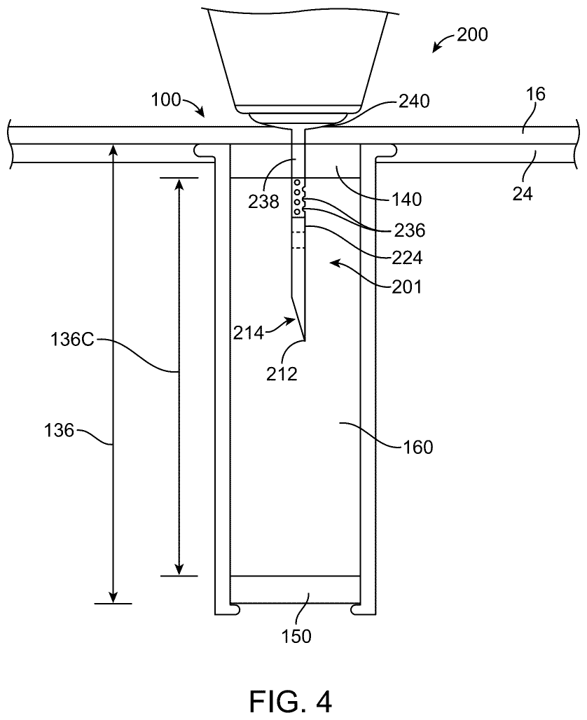

Initial filling of the device 100 with one or more therapeutic agents can occur prior to insertion or after insertion of the device 100 in a patient's eye. Refilling of the device 100 with additional therapeutic agent following a period of sustained treatment can be performed while the device 100 remains within the patient's eye. FIG. 2 illustrates a fluid exchange needle device 200 having a proximal hub or connector 290 and an elongate needle structure 201 having a working length projecting from the connector 290. The connector 290 is configured to attached to a corresponding connector 320 of a syringe 300 or other injection apparatus configured to contain the therapeutic agent formulation 260 in a container 310 and inject the formulation 260 from the container 310 into the implant device 100. The elongate needle structure 201 is designed for insertion through the septum 140 of the implanted device 100. The septum 140 as well as the encasement 110 can be penetrated with the needle structure 201. The encasement 110 and the septum 140 can be penetrated during filling and/or refilling of the reservoir 160. The needle structure 201 of the fluid exchange needle device 200 can be inserted through the septum 140 until a distal opening 214 of the needle structure 201 enters the reservoir 160 (see FIG. 4).

The encasement 110 and the septum 140 can be configured to reseal after penetration during filling and/or refilling of the reservoir 160 and upon removal of the needle structure 210 from the device 100. The septum 140 can reseal around the needle track or path created by the needle structure 201 upon its removal. The device 100 can be periodically refilled with therapeutic agent following surgical placement as needed by accessing the implanted device 100 and without necessitating device removal. The conjunctiva 16 can be lifted or incised away. Alternatively, the conjunctiva 16 can be pierced with the needle structure 201 such that a single penetration in the eye is performed (i.e., single penetration through each of the conjunctiva 16, encasement 110, and septum 140.

Again with respect to FIG. 4, the needle structure 201 can be sized so as to place the distal tip 212 of the needle structure 201 at a location within the reservoir 160 of the implanted device 100 when the surface of the stop 240 is positioned against the conjunctiva 16, for example. The length of the needle structure 201 between the stop 240 and the distal tip 212 can be a percentage of the total length 136 of the implantable device 100, and in some implementations no more than about half of the length 136C of the reservoir 160. A plurality of openings or side ports 236 positioned along the length of the needle structure 210 can be positioned within the reservoir 160 near the septum 140 of the implant 100 in order to receive pre-existing fluid from the reservoir 160 simultaneously as the new therapeutic agent is injected into the reservoir 160. The extension 238 extends substantially through the septum 140, for example, at least about half way through the septum 140 so as to position the side ports 236 away from an external surface of the septum 140 and to inhibit leakage. Fluid exchange of an implanted device 100 is described in more detail in U.S. Pat. Nos. 9,883,968 and 11,419,759, which are incorporated by reference here.

The therapeutic devices described herein can be implanted in the eye to treat the eye for as long as is helpful and beneficial to the patient. For example the device can be implanted for at least about 1 month, 2 months, 3 months, 6 months, 1 year, 2 years, 3 years, 4 year, 5 years and up to permanently for the life of the patient. Alternatively, or in combination, the device can be removed when no longer helpful or beneficial for treatment of the patient. In other implementations, the device can be implanted for at least about 4 years to 10 years, for example a duration of treatment period for a chronic disease such as diabetic macular edema or age-related macular degeneration. The device can be periodically refilled in the physician's office with new therapeutic agent as indicated by disease progression. For diseases such as age-related macular degeneration, the device can be refilled as frequently as once every week, bi-weekly, monthly, bi-monthly, every 3 months, every 4 to 6 months, every 3 to 9 months, every 12 months, or any other period as indicated to treat a disease.

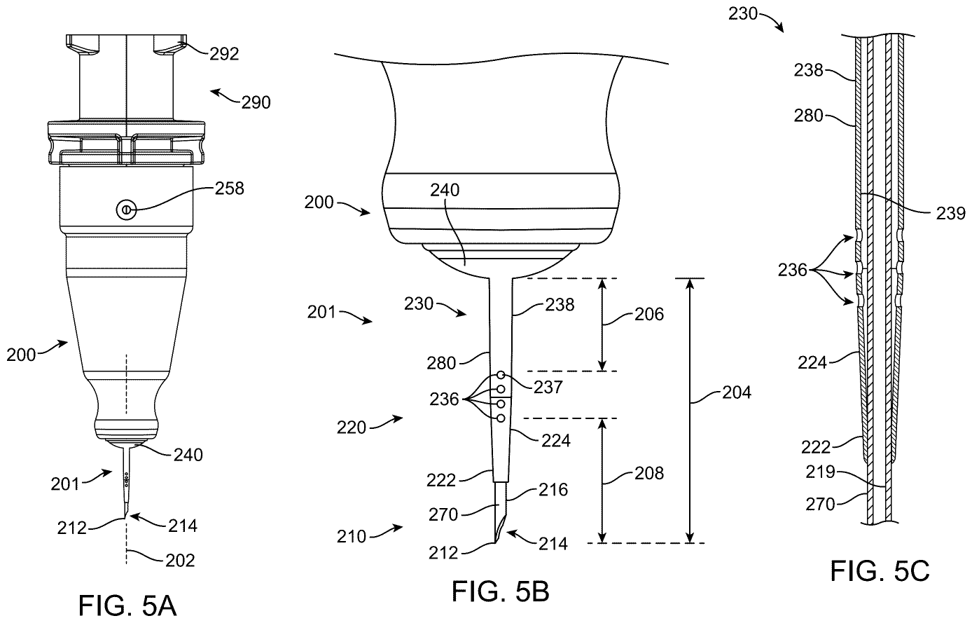

FIG. 5A is a side view of an exchange needle device 200 for refilling of an implanted therapeutic device 100. FIG. 5B is a cross-sectional view of the exchange needle device 200. FIG. 5C is a detail view of an elongate needle device 200 of FIG. 5A. FIG. 5D is a cross-sectional view of a segment of the elongate needle structure 201 of FIG. 5A. The device 200 can be coupled to or include on a proximal end region a syringe 300 having a container 310 to inject a therapeutic fluid into the reservoir 160 of the device 100. The device 200 can include an elongate needle structure 201 having a working length projecting from the proximal hub or connector. The working length of the needle structure 201 can be placed substantially within at least a portion of the device 100.

Still with respect to FIGS. 5A-5C, the needle structure 201 can have a distal portion 210, an intermediate portion 220, and a proximal portion 230.

The distal portion 210 can include a distal tip 212 configured to penetrate the septum 140 of the implantable device 100. The at least one opening 214 to inject therapeutic fluid into the implantable device 100 can be found at or near the distal tip 212. The intermediate portion 220 can include a tapered section 224 that gradually increases a size of the channel formed in the septum 140 when the needle structure 201 is advanced through the septum 140 so as to maintain integrity of and mitigate damage to the septum 140. The tapered portion 224 can extend along axis 202 and can be without holes so as to decrease pressure to the septum 140 that may otherwise occur near the edge of a hole.

The needle structure 201 can include at least one opening 214 to place the therapeutic fluid in the reservoir 160 of the device 100 and a plurality of openings or side ports 236 extending through a wall of the outer cannula 280 to receive the fluid from the reservoir 160 of the implantable device 100 into an annular space between the inner and outer cannulae. The distal end of the inner cannula 270 can include a sharpened beveled tip forming the opening 214.

The proximal portion 230 can include the plurality of side ports 236 to receive the fluid from the reservoir 160 of the implantable device 100. The needle structure 201 can include a stop 240 to limit a depth of insertion of the needle structure 201 into the reservoir 160 of the device 100, for example, no greater than about 4.5 mm. The stop 240 can be a deformable material to engage with the tissue during injections. The proximal portion 230 can include an extension 238 extending from the stop 240. The extension 238 can be without holes to inhibit leakage when the fluid is exchanged and the stop 240 engages the conjunctiva 16.

When coupled to the therapeutic device 100, the stop 240 can be positioned to engage the conjunctiva 16 and the needle structure 201 can extend through the conjunctiva 16 and the septum 140 into the device 100. The needle structure 201 can be sized so as to place the distal tip 212 at a location within the device 100 when the surface of the stop 240 contacts the conjunctiva 16, for example. The distal tip 212 can be located on the needle structure 201 so as to place the distal tip 212 at a location from the septum 140 within the device 100 that is no more than a desired length, such as about ¾ of the length of the implantable device 100, and in some implementations no more than about half of the distance of the device 100. The extension 238 can extend substantially through the septum 140, for example, at least about half-way through the septum 140 so as to position the plurality of openings away from an external surface of the septum 140 and to inhibit leakage.

FIG. 5B shows a detail view of the elongate needle structure 201 of the exchange needle device 200 of FIG. 5A having a working length projecting from the proximal hub. The needle structure 201 extends along axis 202 between the distal tip 212 and the stop 240. The distal portion 210 can include an extension 211 having a substantially constant cross-sectional size extending between the tip 212 to penetrate tissue and the intermediate portion 220. The needle structure 201 can include an inner cannula 270 (which may be referred to herein as an elongate tube or a fill cannula or a needle) extending distally from the proximal hub or connector 290. The inner cannula 270 has an outer surface, a proximal end, a distal end 212, and an inner surface defining a bore or channel 219 that extends to a distal opening 214 at a distal end 212 of the inner cannula 270. The elongate needle structure 201 also includes an outer cannula 280 or sheath extending distally from the proximal hub 290. The outer cannula 280 also includes an outer surface, a proximal end, a distal end, and an inner surface defining a bore extending to a distal opening at the distal end of the outer cannula 280. The inner cannula 270 can extend through the bore or internal lumen of the outer cannula 280 such that the outer cannula 280 surrounds at least a proximal end region of the inner cannula 270. The inner diameter of the outer cannula is sized relative to the outer diameter of the inner cannula 270 so as to form an annular space between the outer surface of the inner cannula 270 and the inner surface of the outer cannula 280. The distal end of the outer cannula 280 is located a distance proximal to the distal opening of the inner cannula 270. The elongate needle structure 201 of the exchange needle device 200 can additionally incorporate a lubricious coating on the outer surface of the outer cannula 280 and the outer surface of the inner cannula 270 extending distal to the distal end of the outer cannula 280. The lubricious coating covers at least a portion of the working length of the elongate needle structure 201, which will be described in detail below.

The distal tip 212 of the inner cannula 270 has at least one opening 214 through which material injected through the lumen of the inner cannula 270 may flow out the opening 214. The distal tip 212 can be sharp or blunted. The outer cannula 280 can be configured to receive preexisting material from the reservoir 130 such that it can be flushed out from the reservoir 160 upon filling with new material through the inner cannula 270. Thus, the outer cannula 280 can include at least one opening into its lumen. The opening can be formed at a distal end of the outer cannula 280 formed between the outer surface of the inner cannula 270 and the inner surface of the outer cannula 280. Alternatively, or in additionally, the opening can include a plurality of openings or side ports 236 through a wall of the outer cannula 280 as described in more detail below.

Still with respect to FIG. 5B, the extension 211 can include a portion of the inner cannula 270 extending from the stop 240 to the tip 212 of the inner cannula 270. The tip 212 can be configured to penetrate tissue, such as the tip of the needle to penetrate conjunctival tissue. The tip 212 and the opening 214 can be located a distance 204 from the stop 240 and the plurality of side ports 236 to provide efficient exchange of the fluid within the reservoir 130 of the implanted device 100. In some implementations, the opening 214 is placed at a distance from the stop 240 greater than the plurality of side ports 236 such that the opening 214 is located distal to the plurality of side ports 236. This relative position between the opening 214 and the plurality of side ports 236 can inhibit mixing of the injected therapeutic fluid moving into the reservoir 160 through opening 214 with the fluid within the implanted device 100 moving out of the reservoir 160 through side ports 236. The opening 214 can be separated from the plurality of side ports 236 by a distance 208, such that the opening 214 can be located below the plurality of side ports 236 when the therapeutic fluid is injected.

The therapeutic fluid may have a density greater than the fluid of the implanted device and opening 214 can be placed below the plurality of side ports 236 when the therapeutic fluid is injected to inhibit mixing of the fluids (i.e., the fluid moving in from the fluid moving out). The axis 100A of the implantable device 100 and the corresponding axis of the reservoir 160 can be oriented away from horizontal, such that porous structure 120 may be located below the septum 140 when the therapeutic fluid is injected. The axis 202 can be oriented away from horizontal such that opening 214 can be placed below the plurality of side ports 236. The therapeutic fluid having the greater density can flow toward the distal end of the therapeutic device and the displaced fluid from the implantable device having the lesser density can be received by the plurality of side ports 236 located above the opening 214. It should be appreciated that inner cannula 270 can be movable relative to the outer cannula 280 or the two components can be in a fixed configuration relative to one another.

Examples of therapeutic agents and corresponding formulations and fluids that may be delivered using the devices described herein are described in the applications incorporated by reference herein and in Table 1 of U.S. application Ser. No. 14/937,784, published as U.S. 2016/0128867, which is incorporated herein in its entirety. Therapeutic agents that can be delivered from the devices described herein include but are not limited to triamcinolone acetonide, bimatoprost or the free acid of bimatoprost, latanoprost or the free acid or salts of the free acid of latanoprost, ranibizumab, travoprost or the free acid or salts of the free acid of travoprost, faricimab, zifibancimig, timolol, levobunalol, brimonidine, dorzolamide, brinzolamide, and others for the treatment of ocular diseases.

The therapeutic agents and corresponding formulations can have a density greater than the density of the fluid within the chamber of the implanted device. For example, one or more of the therapeutic agent or a stabilizer can increase the density of the therapeutic fluid. In many embodiments the therapeutic fluid having the greater density comprises a stabilizer, such as trehalose, and the therapeutic agent such as a protein comprising an antibody fragment. Alternatively or in combination, the therapeutic formulation can include an amount of therapeutic agent sufficient to provide a density greater than the fluid of the implanted device. The difference in density can be within a range from about 1% to about 10% and can depend on the density of the fluid within the reservoir chamber of the therapeutic device and density of the therapeutic fluid placed in the reservoir chamber with the exchange apparatus. The density of the therapeutic fluid may correspond to a density of the therapeutic agent and a density of the stabilizer (when present). In many embodiments, the density of the fluid of the reservoir chamber may correspond to a density of phosphate buffered saline, or plasma, or an amount of therapeutic fluid remaining in the reservoir from a prior exchange, or combinations thereof, for example. As described elsewhere herein, differences in fluid density as a result of temperature differences between the exchanged fluids can improve bottom-up filling efficiency. As mentioned above, implant orientation and/or tilt angle between the implant and the exchange needle during refilling can improve refill efficiency where there is a solution density different between the fluid being injected and the contents of the implant. Aspiration can be incorporated to aid in the efficiency of the exchange as well.

When injected into a device 100 implanted within the patient, the distance 204 may correspond to no more than approximately the length of the device 100. The distance 204 may be substantially the length of the reservoir 160 so as to place the distal tip 212 near, but not touching the porous structure 120, and the needle structure 201 of the exchange needle device 200 can be aligned with an elongate axis 100A of the implantable device 100. In many embodiments, the distance 204 may correspond to no more than about half the distance of the reservoir 160 such that the needle structure 201 can be readily aligned with the implantable device. Work in relation to embodiments suggests that a distance providing a tolerance for angular alignment error of the axis 100A with the axis 202 can facilitate exchange and improve efficiency of the exchange. The distance 204 from stop 240 to tip 212 can be no more than about half of the axial distance of the implantable device can facilitate alignment during injection.

The intermediate portion 220 can include an extension 222 extending between the tapered portion 224 and the distal portion 210 (see FIGS. 5B-5C). The extension 222 can have a cross-sectional size that is smaller than the tapered portion 224. The extension 222 can have a smooth outer surface to penetrate tissue. The tapered portion 224 can have a smooth outer surface to penetrate tissue and the septum 140 of the device 100. The outer surface of the tapered portion 224 can extend at an angle of inclination relative to the axis 202, and the tapered portion 224 can include a conic section having an angle with the axis such that the outer surface extends at the angle of inclination relative the axis. The angle of inclination of the tapered portion 224 can be no more than about 25 degrees, for example. The angle of inclination can be about 1 degree, about 2 degrees, about 5 degrees, about 10 degrees, about 15 degrees, about 20 degrees, or about 25 degrees, for example. The extension portion 216 (i.e., the distal end region of the inner cannula 270 extending distally past the distal end of the outer cannula 280) can have a first cross-sectional dimension, and the portion of the outer cannula 180 having the plurality of side ports 236 can have a second cross sectional dimension greater than the first dimension, such that tapered portion 224 having the angle of inclination extends therebetween to connect the extension portion 216 with the portion having the plurality of side ports 236.

Still with respect to FIG. 5B, the proximal portion 230 can include the plurality of openings or side ports 236 spaced apart along the axis 202 and distributed circumferentially around a longitudinal axis of the proximal portion of the outer cannula 280 to receive fluid from a plurality of circumferential and axial locations when the stop 240 engages the conjunctiva 16 to place the plurality of openings within the reservoir chamber. At least one side port 237 of the plurality of side ports 236 extending through a wall of the outer cannula into the annular space between the inner and outer cannulae can be separated from the stop 240 with a distance 206 corresponding substantially to the thickness of the septum 140, such that the at least one side port 237 of the plurality of side ports 236 can be placed near the inner surface of the septum 140 to receive fluid contacting the inner surface of the septum 140. In some implementations, the height of the septum 140 from the lower surface 142 to the upper surface 144 is within a range from about 0.25 to about 2 mm, about 0.5 to about 1.5 mm, or about 1.0 mm to about 1.3 mm, preferably about 1.1 mm to about 1.2 mm, such that the thickness of the septum 140 is substantially greater than a thickness of the conjunctiva which can be approximately 100 μm. The distance 206 corresponding substantially to the thickness of the septum 140 can correspond substantially to the thickness of the septum 140 and the epithelium of the patient.

As mentioned, the outer cannula 280 can be configured to extend over at least a portion of the inner cannula 270. The outer cannula 280 can extend along the intermediate portion 220 and the proximal portion 230, and the inner cannula 270 can extend through the outer cannula 280. The outer cannula 280 can include the plurality of side ports 236 and provide one or more channels extending along inner cannula 270 to pass the fluid of the implantable device through the septum 140.

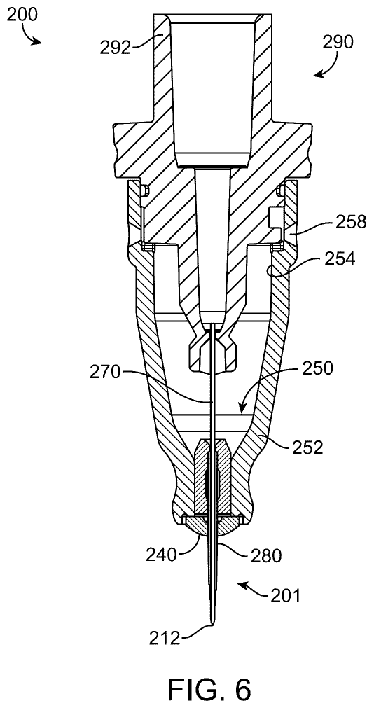

FIG. 5C illustrates a cross-sectional view of a needle structure 201 of the exchange needle device 200 having the outer cannula 280 extending over the inner cannula 270. The inner cannula 270 can include a channel 219, for example a bore or lumen, extending distally to the distal opening 214 at a distal end region of the inner cannula 270 and proximal to a connector to couple the channel 219 to a syringe 300 holding the therapeutic fluid 260 to be injected into the device 100. The outer cannula 280 can include portions corresponding to the intermediate and proximal portions of the needle structure 201. FIG. 5C shows the extension 222 of the outer cannula 280 having an inner surface sized to engage an outer surface of the inner cannula 270. The inner diameter of the outer cannula is smaller at the distal end compared to an inner diameter of the outer cannula near the proximal end. The inner diameter of the outer cannula at the distal end contacts an outer diameter of the distal end of the elongate tube. In some implementations, the diameter of extension 222 can have an inner diameter that approaches an outer diameter of the inner cannula 270 to engage the inner cannula 270 with at least one of pressure or friction such that substantially no annular space exists between them at the distal-most end of the outer cannula 280. This minimizes distal-facing space between the inner cannula 270 and the outer cannula 280 that can contribute to coring of the septum 140 upon insertion of the needle structure 201 into the device 100. The tapered portion 224 can include an intermediate portion of outer cannula 280, in which the outer cannula 280 has a tapered surface to penetrate the tissue and septum 140. The proximal portion 230 can include a proximal portion of the outer cannula 280 having the plurality of side ports 236 and the extension 238. An annular channel 239 can extend along an outer surface of the inner cannula 270 to the plurality of side ports 236. The annular space or channel 239 extending between the cannulae 270, 280 is located a distance proximal to the distal-most end of the outer cannula 280 and begins near the location of the tapered section 224 and the flaring outward of the outer cannula 280 away from the inner cannula 270 near the proximal portion 230 and the location of the side ports 236. The tight tolerance between the inner diameter of the outer cannula 280 at the location of the extension 222 and the outer diameter of the inner cannula 270 at the location of the extension 222 reduces coring and damage of the septum 140 upon insertion of the needle structure 201 into the device. The channel 239 can extend proximally along extension portion 238 toward a collection chamber 250 to receive the fluid of the implantable device 100 (see FIG. 6). The channel 239 can couple the plurality of side ports 236 to the collection chamber 250 to receive the fluid of the implantable device 100.

It should be appreciated that the tapered portion 224 can change in outer diameter according to a variety of geometries and use of the term “taper” or “tapered” is not intended to be limiting to any particular shape or geometry.

Generally, the needle structure 201 can have a larger outer diameter at a proximal end region of the tapered section 224 and a smaller outer diameter at a distal end region of the tapered section 224. The change in outer diameter can, but need not be constant along an axial length of the tapered section 224. For example, the tapered section 224 can incorporate one or more cylindrical regions between the proximal-most end of the tapered section 224 and the distal-most end of the tapered section 224. The tapered section 224 can be a linear taper or conical in shape or can be curved or have the shape of an arc or ogive.

As mentioned above, the exchange needle device 200 can include or be configured to couple to a syringe 300 or other container configured to hold fluid to be delivered to the reservoir 160. FIG. 6 shows an implementation of an exchange needle device 200 having a proximal hub or connector 290 configured to couple to a syringe 300. The connector 290 can be a locking connector having an extension 292 sized to fit in a channel of connector 320 of syringe 300. The exchange needle device 200 can include components of a standard locking needle assembly, for example a standard locking needle such as a Luer-Lok™ fitting or a pressure fit connector. Alternatively, the connector 290 may include a non-standard connector to limit access to the exchange needle device 200. For example, the connector 290 can be a star connector or other connector, and connector 290 may include a lock and key mechanism. The lock and key mechanism can have a lock on the exchange needle device 200 configured to receive a key of the injector, such that the lock of connector 290 can receive the key of connector to couple the injector to the exchange needle device 200 and permit injection from chamber through opening 214. Alternatively, the syringe may be affixed to exchange needle device 200, and syringe provided with a single dose of therapeutic agent.

The exchange needle device 200 also includes a collection chamber 250 configured to receive fluid from the reservoir 160. The collection chamber 250 can be defined by a wall 252 configured to surround the inner cannula 270 extending through the outer cannula 280. The wall 252 can extend a substantial distance from the stop 240 and can include at least one opening 258 that can vent to atmospheric pressure. An outlet channel 254 can extend from container 250 to the at least one vent opening 258 to atmospheric pressure.

Again with respect to FIGS. 5A-5C, the outer cannula 280 can be configured in many ways and can have a wall thickness from about 0.250 μm to about 250 μm, for example about 25 μm (0.001 inches or 1/1000 inch). The outer cannula 280 can include an inside diameter sized larger than the outside diameter of inner cannula 270 so as to provide an annular channel 239 extending axially between the inner cannula 270 and the outer cannula 280 from the plurality of side ports 236 to the opening 285. The inner cannula 270 can be as large as about 20 gauge (maximum OD of about 0.92 mm), although smaller sizes are preferred, for example, 30 gauge or 34 gauge and are received within suitably larger outer cannula 280 (e.g., 28 gauge). The size of the inner and outer cannulae are limited by the diameter of the septum 140 being penetrated. The elongate needle structure 201 formed by the inner and outer cannulae as a maximum outer diameter that is preferably no greater than about 50% the size of the septum diameter. For example, a septum of about 1.1 mm in diameter can receive an elongate needle structure 201 that is as large as about 25 gauge (maximum outer diameter of 0.53 mm), although smaller sizes are preferred. The diameter of each of the side ports 236 can be within a range from about 2.5 mm down to about 3 microns, for example within a range from about 25 microns to about 250 microns, or about 50 microns to about 150 microns. The diameter of each of the plurality of side ports 236 can be uniform or can vary in size as well as shape.

The plurality of side ports 236 can be one or more of many shapes and can be arranged in many ways. The side ports 236 can be arranged in columns of holes, where each column can include from about 1 to about 20 holes, for example, and can be circular, oval, elliptical or other shapes, for example. The outer cannula 280 can include four columns of circular holes forming the side ports 236. Each of the side ports 236 can have a diameter of no more than about one half of the thickness of the outside diameter of the outer cannula 280, for example, and may be located circumferentially at 90 degrees to each other, for example. Each of the four columns may extend axially along the outer cannula 280. The columns can be spaced angularly at 90 degrees to each other, for example. The outer cannula 280 can include two columns, each column comprising about four side ports 236, each side port 236 having a diameter of no more than about one eighth of the diameter of the outside diameter of the outer cannula 280. The two columns may be spaced apart circumferentially at 180 degrees, and the side ports 236 can include holes cross-drilled through both sides of the outer cannula 280, such that each hole has a corresponding hole on the other column on an opposite side of the sheath. The outer cannula 280 can include about four cross-drilled holes, each hole having a diameter of no more than about three quarters of the diameter of the outside diameter of the outer cannula 280, for example. The holes can be pairs of holes, in which the holes of each pair have corresponding axial locations. The holes can be arranged in two columns spaced circumferentially at 180 degrees. The outer cannula 280 can include at least about three columns of at least about 3 holes, each hole having a diameter of no more than about one quarter of the diameter of the outside diameter of the outer cannula 280. The columns can be spaced apart circumferentially at about 120 degrees, for example. The outer cannula 280 can include at least about 40 side ports 236, each side port 236 having a diameter of no more than about one tenth of the diameter of the outside diameter of the outer cannula 280.

The arrangement of the opening 214 from the inner cannula 270 can vary as well. For example, the opening 214 can be configured to change direction of flow from the inner cannula 270 into the reservoir 160 to impact refill efficiency. The opening 214 can include one or more side openings located near the distal tip 212 of the inner cannula 270. The side ports 236 in the outer cannula 280, the openings 214 in the inner cannula 270, the density/viscosity of the therapeutic fluid being injected, the presence of one or more flow director type features within the device 100 can all impact the effective flow patterns within the device to improve exchange efficiency.

Again with respect to FIG. 6 showing a cross-sectional view of the fluid exchange needle device 200 for coupling to a syringe 300. The channel 239 extends from the plurality of side ports 236 to a container 250 to receive the fluid of the implanted device 100. The distal portion 210 has an opening 214 at the distal tip 212. The channel 219 extends along an axis 202 from the opening 214 to a connector 290. The connector 290 is configured to couple to the connector 320 of the syringe 300. The syringe 300 (or other injector type device) can include a container 310 having the therapeutic fluid to be injected into the device 100. The container 310 can be fluidically coupled to the opening 214 on the distal tip 212 when the connector 320 engages the connector 290.

Again with respect to FIG. 6, the collection chamber 250 of the exchange needle device 200 can have a volume (e.g., no more than about 200 uL, or no more than about 150 uL, or no more than about 100 uL, or no more than about 50 uL). A porous structure can be located within at least a region of the collection chamber 250 along the vent path or the vent opening 258 can be open without any porous structure. The porous structure can be formed of a material having a low resistance to air and other gasses while substantially inhibiting flow of a liquid, such as the liquid from the device 100.

### Lubricious Coating

Penetration of the septum 140 by the exchange needle device 200, particularly over time and after repeated penetrations, can impact the integrity of the septum 140 and the extended life of the implant 100. For example, if there is an annular space between the inner cannula 270 and outer cannula 280 at a distal end region of the needle structure, penetration of the septum 140 by the exchange needle device 200 can contribute to coring of the septum 140. The pressure and/or friction of the exchange needle apparatus penetrating the septum 140 can also impact retention of the septum 140 within the proximal end region of the device 100 desired for long-term treatment.

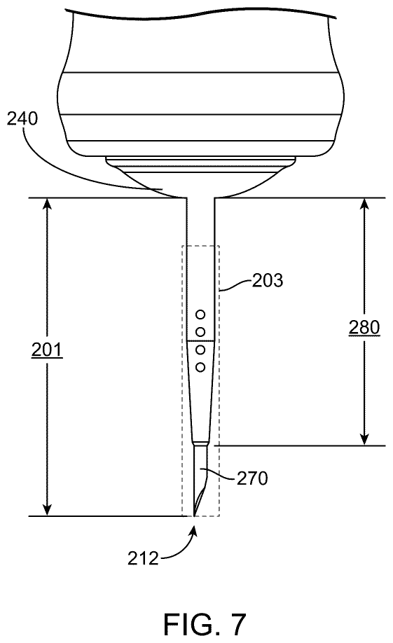

As mentioned above and as shown in FIG. 7, the elongate needle structure 201 of the exchange needle device 200 can incorporate a lubricious coating 203 along at least a portion of its working length to reduce friction and thus insertion force during penetration of the septum 140. The lubricious coating 203 can be on the outer surface of the outer cannula 280 and the outer surface of the inner cannula 270 extending distal to the distal end of the outer cannula 280. FIG. 7 illustrates the needle structure 201 including the outer cannula 280 surrounding a proximal end region of the inner cannula 270 and the distal tip 212. The total length of the needle 210 from the stop 240 to the distal tip 212 can be about 4.0 mm to about 5.0 mm. The outer cannula 280 can have a length that is about 4 mm from the stop 240 to its distal-most end. The inner cannula 270 can extend a distance past the distal-most end of the outer cannula 280 so that its distal tip 212 is available for penetration of the septum 140. The coating 203 can extend from the distal tip 212 up to a region just short of the location where the needle structure 201 extends outside the stop 240. The coating 203 of the needle structure 201 contributes to improved septum retention with repeated penetrations.

In some implementations, the coating 203 covers the entire working length of the needle structure 201 (i.e., the length of the needle structure 201 extending distal to the stop 240). In other implementations, the coating 203 avoids covering the entire working length of the needle structure 201. The portion of the working length of the needle structure 201 covered by the coating 203 includes at least the distal-most end of the needle structure 201 up to about 2 mm from the distal-most end. Preferably, the coating 203 covers the needle structure 201 from the distal-most end at least about 2 mm up to and no greater than about 5 mm from the distal-most end of the needle structure 201 so that about 5% of the total working length is left uncoated. The coating 203 can cover at least about 50% of the total working length of the needle structure 201 starting at the distal-most end up to about 95% of the total working length of the needle structure 201 starting at the distal-most end. For example, if the total working length of the needle structure 201 is 5 mm between the distal stop 240 to the distal-most end of the tip 212, a coating 203 that covers 50% of the total working length covers 2.5 mm of the distal end region of the needle structure 201. The length of the coating 203 can vary depending on the length of the device to be refilled and the working length of the needle structure 201. The coating 203 can extend along at least a portion of the working length of the needle structure 201 from about 3.5 mm up to about 4.5 mm from the distal-most tip 212 of the inner cannula 270 up to a region near the stop 240, or from about 4.0 mm to about 4.25 mm. The lubricious coating 203 can extend over the side wall of the outer cannula at a level of the at least one opening and terminate a distance from the proximal hub. The coating 203 along the side wall of the outer cannula 280 can terminate a distance away from the proximal hub 290, such as about 0.25 mm to about 2 mm away. The length covered by the lubricious coating is generally less than an exposed length of the elongate tube and outer cannula relative to the proximal hub.

The coating 203 can coat the side wall of the inner cannula 270 where the inner cannula 270 extends distal to the outer cannula 280. The coating 203 can coat the side wall of the outer cannula 280 along an axial distance of the side wall includes the region of the outer cannula 280 through which the side ports 236 extend. The coating 203 can extend over the region of the outer cannula 280 at the location of the side ports 236 without covering the side ports 236 themselves. Rather, the coating 203 covers the side wall of the cannula 280 between the side ports 236.

The lubricious coating 203 covering at least a portion of the working length of the elongate needle structure can be a silicone material, including silicone oils and partially cross-linked silicone materials with plasma polydimethylsiloxane (PDMS) or perfluoropolyethers. The silicone material can be applied to the portion of the working length of the needle structure 201 using a solvent carrier (e.g., TECHSPRAY Precision V 371DE). The lubricity is derived from the silicone and the solvent carrier is not present in the coating 203. The coating 203 covers the external surface of the needle structure 201 while maintaining the patency of the side ports 236 and distal opening 214. The low surface tension of the carrier solvent can result in some of the silicone material covering interior regions of the cannulae. However, the patency of the side ports 236 and the distal opening 214 can be maintained by applying a positive pressure through the cannulae during or after coating, which will be described in more detail below.

The silicone material can be at a concentration in the solvent that is less than about 10%, preferably in a range of 4-7% w/w in the solvent carrier. The silicone material can have a viscosity in a range of about 1,000 cS to about 13,000 cS.

The coating 203 can be applied by dip-coating the distal end of the needle structure 201 into a volume of the silicone material. The dip depth, insertion rate, removal rate, dwell time within the bath, and other parameters can all be controlled. The dip depth can be just 2.5 mm up to about 3.5 mm. The insertion rate can be 100 to about 3,000 mm/min. The removal rate can be 100 to about 3,000 mm/min. The dwell time within a volume of the silicone material (referred to herein as a “bath”) can be about 0 to about 3 seconds.

During and after the dip coating process, the inner and outer cannulae 270, 280 can be purged with a gas to maintain patency of the cannula openings (e.g., side ports 236 and distal openings). The inner cannula 270 can be purged with a positive pressure during advancing into a bath, after advancement into the bath and before withdrawal, while withdrawing from the bath, or after withdrawal of the inner cannula 270 from the bath. The outer cannula 280 can be purged with a positive pressure while advancing into a bath, after advancement into the bath and before withdrawal, while withdrawing from the bath, or after withdrawal of the inner cannula 270 from the bath. The purge of the inner cannula 270 (whether purged during advancement, after advancement, during withdrawal, or after withdrawal) can occur at the same time as the purge of the outer cannula 280 or at a different time as the purge of the outer cannula 280. For example, the purge of the inner cannula 270 can occur while advancing the needle structure into the bath and the purge of the outer cannula 280 can occur after during or after the withdrawal of the needle structure from the bath. Any of a variety of combinations are considered herein. The positive pressure through the bore of the inner cannula and/or the outer cannula can be supplied as an amount of nitrogen, argon or air. For example, positive pressure can be applied through the bore of the inner cannula 270 during insertion of the needle structure into the bath. The positive pressure applied through the bore of the inner cannula 270 can be about 0.165 psi up to about 0.265 psi. Positive pressure can be applied through the bore of the outer cannula 280 after withdrawal of the needle structure 201 from the bath. The positive pressure applied through the bore of the outer cannula 280 during withdrawal can be about 15 psi up to about 200 psi.

The solvent carrier can be evaporated under infrared light to leave the silicone coating on the needle structure 201. Other drying modalities are considered herein as known in the art.

The coating 203 can be plasma treated to at least partially cross-link and immobilize the silicone to the dipped length of the needle structure 201.

Partial cross-linking of the silicone improves the durability of the lubricant coating 203 and reduces issues with silicone migration and/or silicone oil droplet formation. Reactive silicone formulations have limited usable life and often require dispersion in flammable solvents to prevent premature curing. The lubricating material coated on the needle structure can be an inert silicone oil dispersed in a non-flammable solvent. After silicone application and solvent evaporation, partial crosslinking of the silicone can be achieved by exposure of the coated surface to atmospheric plasma using argon. The plasma process can operate at atmospheric pressure. The plasma treatment time can be 0.3 seconds up to about 3 seconds. Unlike chemical crosslinking lubricants, silicone/solvent solution is non-reactive and does not expire.

The lubricious coating 203 on the insertion length of the needle structure 201 reduces the insertion force needed for the insertion length of the exchange needle device to penetrate the septum compared to the insertion force for a corresponding exchange needle device without the lubricious coating 203. The lubricious coating on the insertion length of the needle structure 201 reduces deformation of the septum 140 of the device during penetration by the insertion length of the needle structure 201 compared to a corresponding exchange needle device without the lubricious coating 203 on the needle structure 201. Unlubricated needles tend to deform the septum 140 by approximately 0.2 mm in a direction of withdrawal as the needle structure 201 is removed from the septum 140. The coating 203 can cut that deformation in half so that the septum is deformed in a direction of withdrawal by just 0.1 mm. This reduction in deformation of the septum 140 by the needle structure 201 reduces stress on the septum and improves septum retention over time. The reduction in insertion force and deformation of the septum 140 during penetration by the coated needle structure 201 prevent separation of the septum 140 from the body 130 of the device 100 during penetration thereby improving septum 140 retention and long-term integrity of the device for sustained treatment. Lower penetration force needed to refill the device also improves user experience during a refill procedure. A low insertion force can help give a user feedback that the refill needle has been correctly targeted in the septum 140 of the implant device 100.

The reduction in insertion force needed to penetrate a septum can be about 15% up to about 65%, typically about 25% to about 60% the insertion force of an unlubricated needle. Insertion force of unlubricated needles to penetrate a septum 140 of an implant device 100 is typically about 0.96 N±0.1N. Lubricated needles having the lubricious coating 203 applied as described herein can have a lower insertion force than unlubricated needles that is about 0.34 N up to about 0.80 N. The insertion force of lubricated needles is preferably less than about 0.70 N, less than about 0.50 N, less than about 0.45 N, or less than about 0.40 N.

### EXPERIMENTAL

### Example 1

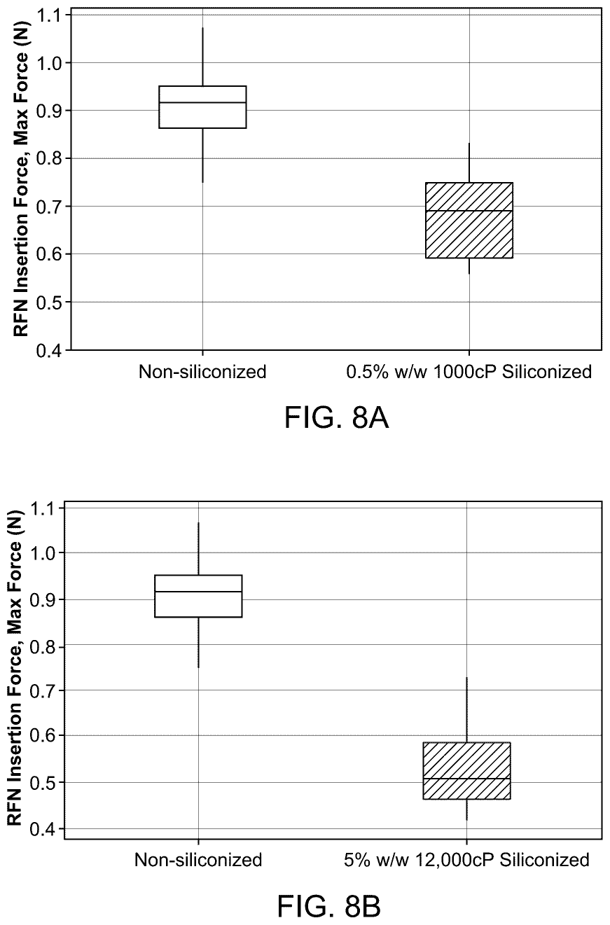

FIGS. 8A-8B are boxplots showing the distribution of insertion force test results for siliconized needles compared to non-siliconized control needles. The Y-axis shows the maximum force in Newtons required to insert the needle structure through the septum into the implant device. FIG. 8A shows low silicone level in which the needle structure was coated with a silicone material having a viscosity of 1,000 cS at 0.5% by weight in solvent. FIG. 8B shows medium silicone level in which the needle structure was coated with a silicone having a viscosity of 12,500 cS at 5.0% by weight in solvent. The insertion force of the control was 0.91 N±0.08 N. The insertion force of low level siliconized needles was 0.68 N±0.09 N (hatched box of FIG. 8A) or about a 25% reduction in insertion force compared to control (open box of FIG. 8A). The insertion force of medium level siliconized needles was 0.53 N±0.08 N (hatched box of FIG. 8B) or about a 50% reduction in insertion force compared to control (open box of FIG. 8B).

### Example 2

The performance testing protocol included as a first step puncturing each implant of each group with a refill needle apparatus and as a second step incubating in phosphate buffered saline (PBS) at an elevated temperature (e.g., 80° C.). As a third step, each implant underwent another puncture at a randomly selected location across the upper surface of the septum. The second and third steps were repeated every 3.5 days until the sample leaks under pressure. Low pressure air was applied with a test apparatus at approximately 0.3 psi to an interior of the implant and leaks assessed by an operator using a microscope to visually inspect the devices submerged in a liquid for continuous formation of air bubbles (e.g., bubbles continue to grow in size over 60 s). Another failure mode includes visual inspection with the unaided eye for missing or dislodged septum. Performance of the implants are described below.

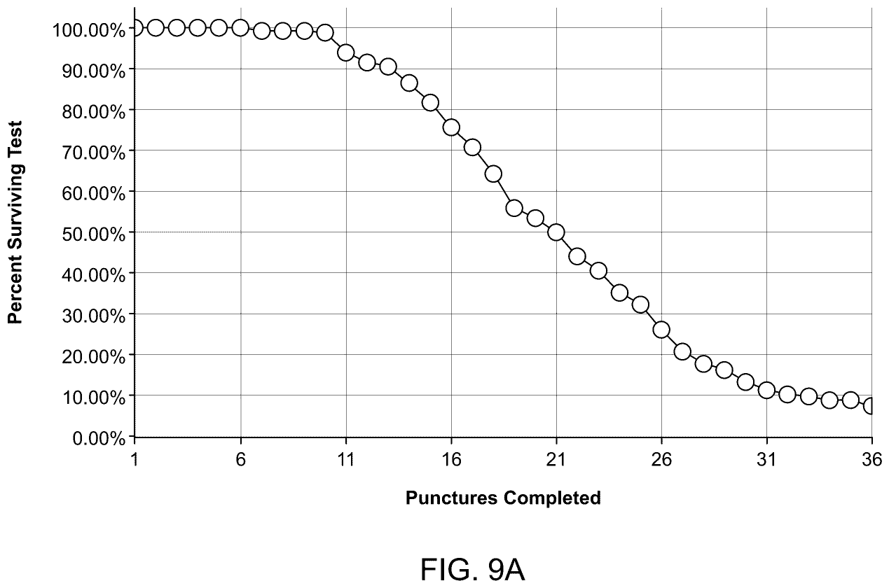

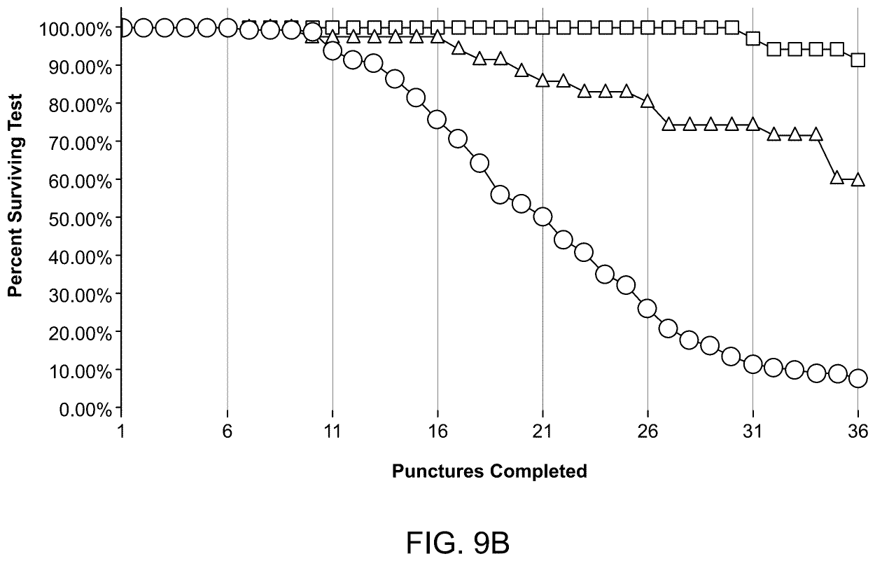

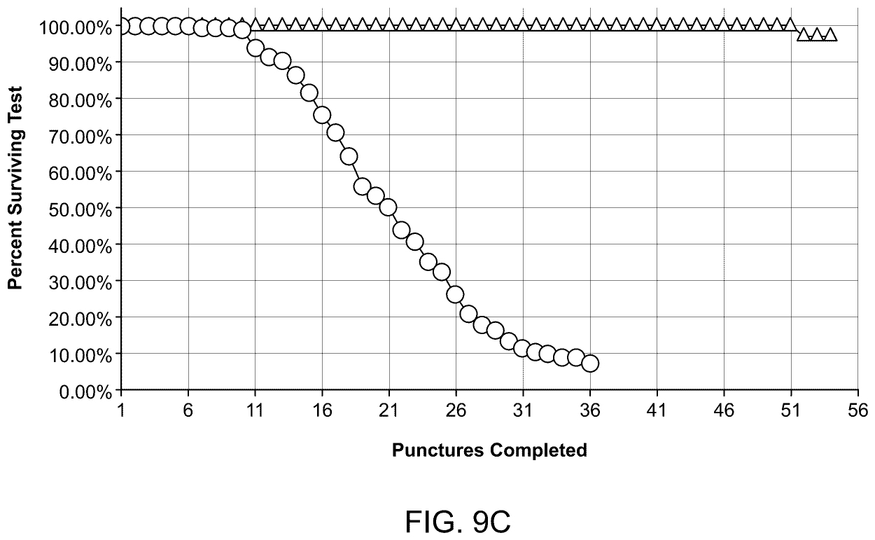

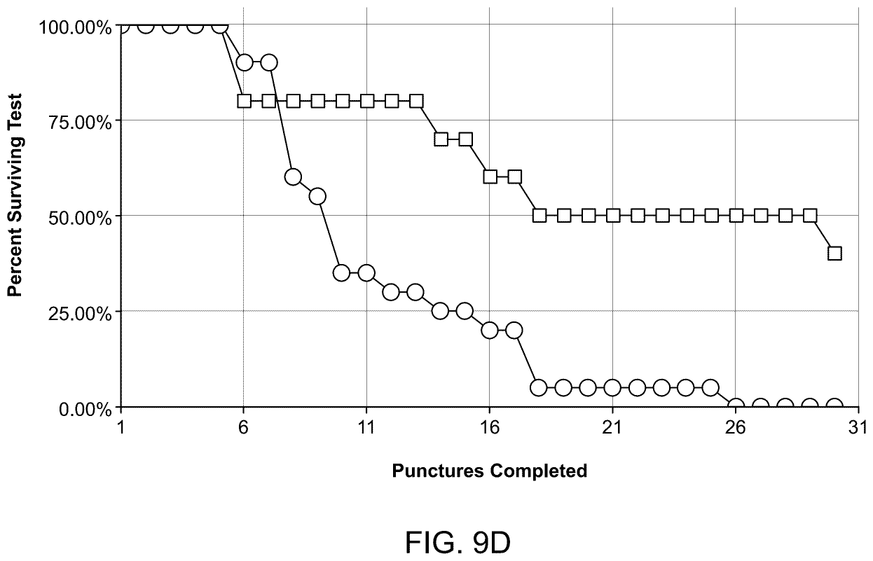

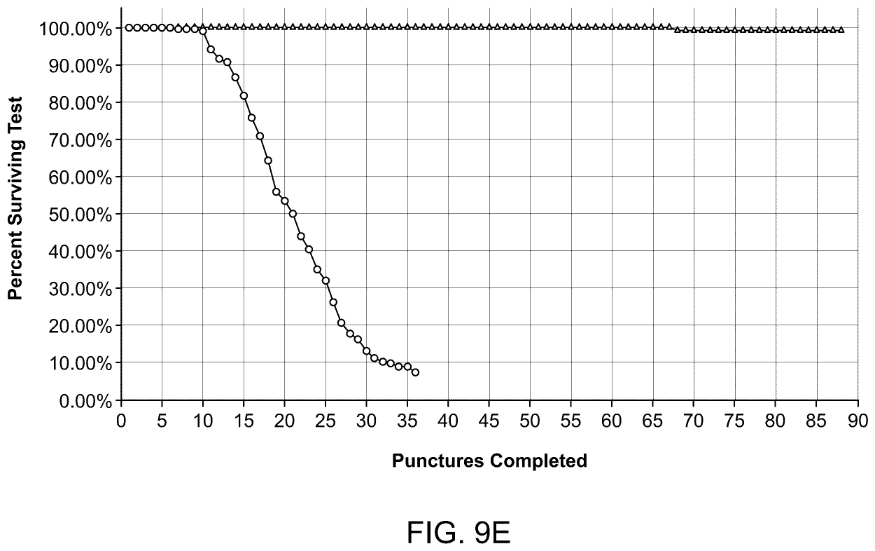

FIG. 9A shows performance of implants after repeated penetration of the septum by an uncoated refill needle. The septum of the implants had an upper surface trimmed only along the central region such that the outer perimeter region remained untrimmed. Further, the untrimmed outer perimeter region of the septum implants lay below the plane of the upper surface of the flange. The trim height of the septum from the lower surface to the planar upper surface at the central region was about 1.14 mm. Approximately 50% of the implants tested (n=188) survived to 21 punctures with the uncoated refill needle. At 36 punctures, which is equivalent of more than about 16 years of implantation and use, less than 10% survived.

FIG. 9B shows performance of implants having the same septum/implant geometry as in FIG. 9A (circles) penetrated by uncoated refill needle compared to implants penetrated by a first refill needle that had a low level of siliconization to reduce insertion force by 25% (triangles) and a second refill needle that had a medium level of siliconization to reduce insertion force by 50% (squares). Low level of siliconization was provided by 0.5% solution of 1,000 cS silicone fluid and medium level of siliconization was provided by 5.0% solution of 12,500 cS silicone fluid. Reducing refill needle insertion force by 25% improved the survival of the implant from 10% as in FIG. 9A to 60% after 36 punctures (n=35). Reducing refill needle insertion force by 50% resulted in a 91% survival through 36 punctures (n=35).

FIG. 9C shows performance of implants having a longer trim height septum Hp that was about 1.22 mm and punctured with siliconized refill needle (triangles) compared to the shorter trim height septum Hc punctured with non-siliconized refill needle (circles) as in FIG. 9A. The siliconized refill needles had 50% less insertion force compared to the unsiliconized refill needles. The implants with the longer septum had double the internal septum bond strength compared to the shorter septum implants. Implant survival at 54 punctures improved from 10% to 97% (n=35).

FIG. 9D shows performance of implants having an upper surface trimmed only along the central region such that the outer perimeter region remained untrimmed and lay below the plane of the upper surface of the flange. The septum trim height of the implants was about 1.05 mm. The survival of implants after puncture with non-siliconized refill needle (circles) and siliconized refill needle (squares) was compared. Implant survival after 18 punctures in the siliconized refill needle group plateaued at about 50% compared to the unsiliconized refill needle group, which had substantially failed by 18 punctures. The siliconized refill need group had about 37% survival after 30 punctures.

FIG. 9E shows performance of implants as in FIG. 9A punctured with nonsiliconized refill needle (circles) compared to improved implants having increased septum contact area, low epoxy, and extra oven curing punctured with siliconized refill needle (triangles). After 36 punctures, which is the equivalent of more than about 16 years of use, the implants in the siliconized refill needle group maintained about 100% survival. The first failure on puncture occurred after 68 punctures. The septum did not dislodge, but appeared to leak through a hole in the septum.

### Example 3

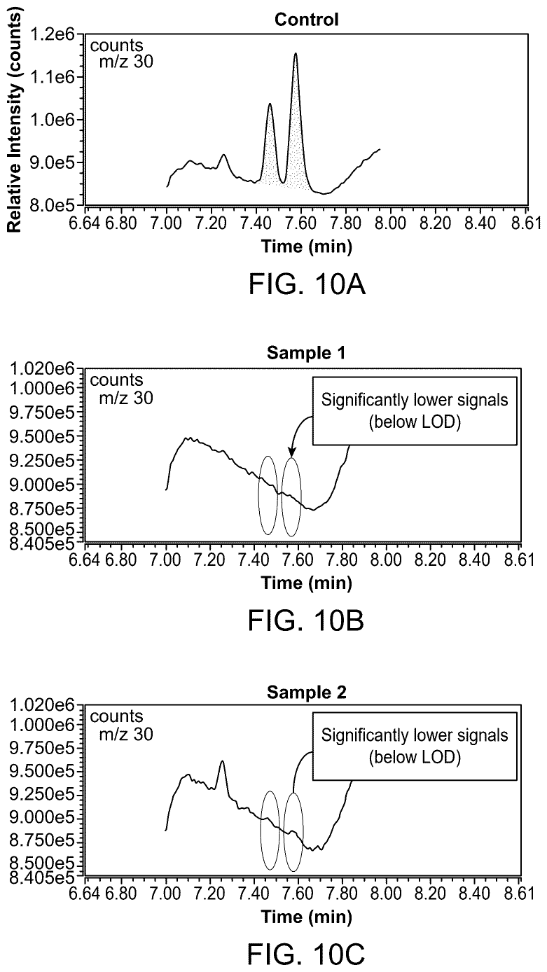

The effectiveness of the second stage of curing on improved septum bonding was demonstrated by measuring residual TMHMD in subassemblies using gas chromatography/mass spectroscopy (GCMS). All subassemblies (i.e., proximal body 130 with bonded septum 140 and no overmolded encasement 110) underwent a first stage of curing of the epoxy (Epotek) dispensed to adhere the silicone septum within the bore of the polysulfone proximal body. The first stage of curing included oven heating bonded subassemblies at 67° C. for 3 hours. A first group of subassemblies underwent no second stage of curing (see control of FIG. 10A). A second group of subassemblies underwent a second stage of curing by oven heating at 150° C. for 3 hours (see Sample 1 of FIG. 10B). A third group of subassemblies underwent a second stage of curing by oven heating at 125° C. for 3 hours (see Sample 2 FIG. 10C). Following curing, five subassemblies of each group were pooled in 0.5 mL methanol in glass vials and agitated at 300 rpm for 30 minutes at ambient temperature. The extracts were tested using GCMS to assess residual 2,2,4(2,4,4)-Trimethyl-1,6-hexanediamine (TMHMD) (CAS-No 25513-64-8 at 60-100% Epoxy part B). The subassemblies of the second and third groups that went through both the first stage of curing and the second stage of curing showed significantly lower signals below LOD (limit of detection or the lowest concentration of the analyte in the test sample that is easily distinguished from zero) for TMHMD residuals within the proximal body compared to control subassemblies (i.e., FIG. 10A) in which only a first stage of curing was performed.

### Example 4

The force necessary to separate an overmolded silicone encasement bonded to an upper surface of a silicone septum epoxied within a bore of a polysulfone body was measured to assess the impact of the second stage of curing on bond strength between the septum and the encasement. Septum/body subassemblies were constructed using only a single stage of curing or both a first stage and a second stage of curing. The first stage of curing included oven heating subassemblies at 67° C. for 3 hours. The second stage of curing included oven heating subassemblies at 115° C. for 0.5 hour, at 115° C. for 3 hours, or 135° C. for 0.5 hour, or 135° C. for 3 hours. Following the cure protocol (whether just one stage of curing or two stages of curing), the elastomeric silicone encasement was overmolded and bonded to the upper surface of the septum. An external region of the encasement encapsulating the flange of the proximal body was cut so that the encasement could be inverted and disengaged from the flange to evaluate the bond between the upper surface of the septum and the lower surface of the encasement alone. The proximal body of the subassembly was held within an implant holding fixture and the inverted encasement clamped and pulled upward away from the upper surface of the septum thereby tensioning the bond between the encasement and the septum. The maximum force required to separate the encasement from the septum was recorded.

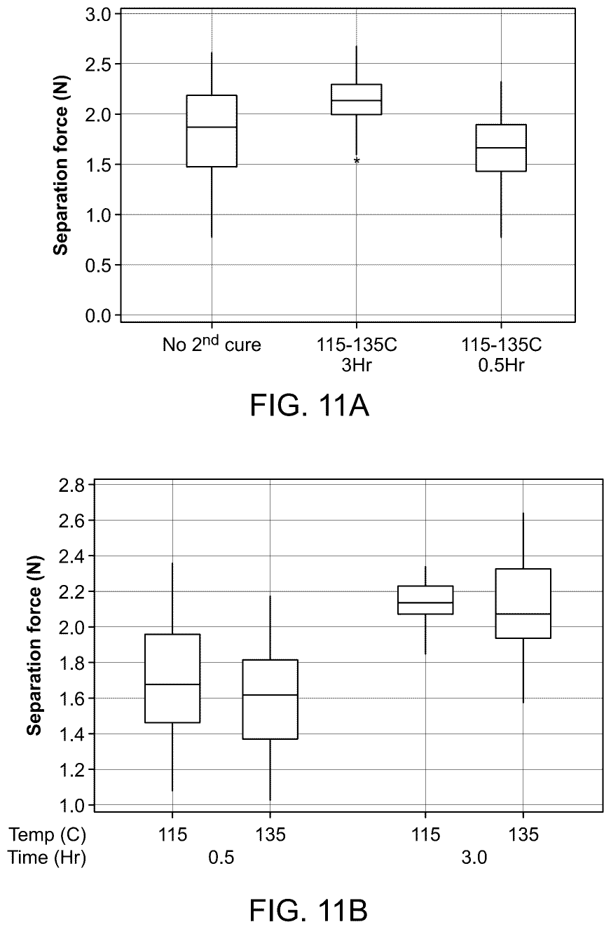

FIG. 11A shows separation force in Newtons (N) for a subassembly without a second stage of curing (see, No 2^{nd} cure) compared to two groups of subassemblies having undergone both a first stage of curing and a second stage of curing. The subassemblies in the first group were heated to 115° C. or 135° C. and results pooled in FIG. 11A. Similarly, the subassemblies in the second group were heated to 115° C. or 135° C. and results pooled in FIG. 11A. The second epoxy cure at the longer period of 3 hours significantly increased the force necessary to separate the overmolded encasement from the upper surface of the septum. The second epoxy cure at the shorter period of time (i.e., 0.5 hour) did not significantly alter the separation force needed to pull the overmolded encasement from the upper surface of the septum.

FIG. 11B shows the effect of temperature and time of the second stage of curing on the separation force in Newtons (N). The second stage of epoxy curing compared 115° C. and 135° C. at 0.5 hour and 3 hours. The subassemblies with a second stage of epoxy curing at the shorter time-period had lower separation force needed to break the bond between the encasement and the septum compared to subassemblies with a second stage of epoxy curing at the longer time-period, even at the higher temperature curing. The length of the second stage of curing rather than the higher temperature increased the robustness of the bond between the overmold encasement and the upper surface of the septum.

### Example 5

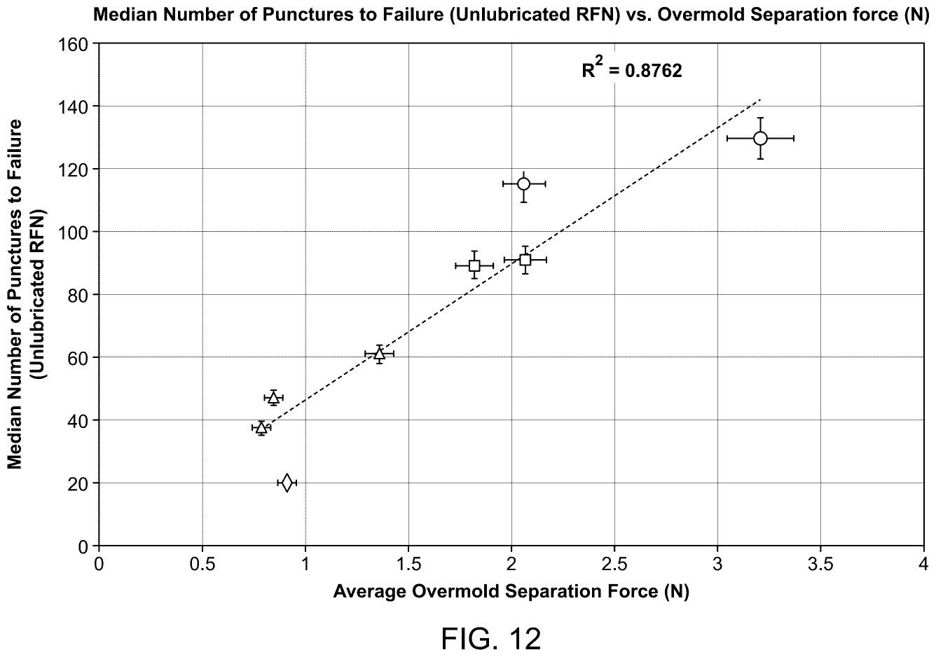

FIG. 12 illustrates the correlation between overmold separation force and punctures-to-failure. The performance testing protocol to assess punctures-to-failure is described above in Example 2. Overmold separation force was measured as described above in Example 4. Overmold separation force in Newtons (N) is shown on the x-axis and punctures-to-failure is shown in the y-axis. Implants having a septum positioned within the bore without using epoxy are shown as filled circles. Implants having an epoxy bonded septum with a fully trimmed upper surface and outer perimeter substantially flush with the upper surface of the flange are shown as filled squares. Implants having an epoxy bonded septum that is shorter and with a partially trimmed upper surface along the central region and an untrimmed outer perimeter countersunk below the upper surface of the flange are shown as filled triangles. Implants having the shortest epoxy bonded septum with a partially trimmed upper surface are shown as filled diamonds. Implants with a higher overmold separation force have higher punctures-to-failure performance (correlation R²=0.8762). Implants having longer septum heights (squares) bonded better than implants with shortest septum heights (triangles and diamonds). Implants without any epoxy bonding the septum to the proximal body had the best outcome.

The devices described herein can be used to deliver essentially any substance. As used herein, “substance,” “drug” or “therapeutic” is an agent or agents that ameliorate the symptoms of a disease or disorder or ameliorate the disease or disorder including, for example, small molecule drugs, proteins, nucleic acids, polysaccharides, and biologics or combination thereof. Therapeutic agent, therapeutic compound, therapeutic regimen, or chemotherapeutic include conventional drugs and drug therapies, including vaccines, which are known to those skilled in the art. Therapeutic agents include, but are not limited to, moieties that inhibit cell growth or promote cell death, that can be activated to inhibit cell growth or promote cell death, or that activate another agent to inhibit cell growth or promote cell death. Optionally, the therapeutic agent can exhibit or manifest additional properties, such as, properties that permit its use as an imaging agent, as described elsewhere herein.

In aspects, description is made with reference to the figures. However, certain aspects may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detain in order to not unnecessarily obscure the description. Reference throughout this specification to “one embodiment,” “an embodiment,” “an aspect,” “one aspect,” “one implementation, “an implementation,” or the like, means that a particular feature, structure, configuration, or characteristic described is included in at least one embodiment, aspect, or implementation. Thus, the appearance of the phrase “one embodiment,” “an embodiment,” “one aspect,” “an aspect,” “one implementation, “an implementation,” or the like, in various placed throughout this specification are not necessarily referring to the same embodiment, aspect, or implementation. Furthermore, the particular features, structures, configurations, or characteristics may be combined in any suitable manner in one or more implementations.

The use of relative terms throughout the description may denote a relative position or direction or orientation and is not intended to be limiting. For example, “distal” may indicate a first direction away from a reference point. Similarly, “proximal” may indicate a location in a second direction opposite to the first direction. Use of the terms “front,” “side,” and “back” as well as “anterior,” “posterior,” “caudal,” “cephalad” and the like or used to establish relative frames of reference, and are not intended to limit the use or orientation of any of the devices described herein in the various implementations.

The word “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples, embodiments, aspects, and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

1. An exchange needle device comprising:
a proximal hub; and
an elongate needle structure having a working length projecting from the proximal hub, the elongate needle structure comprising:
an elongate tube extending distally from the proximal hub, the elongate tube comprising an outer surface, a proximal end, a distal end, and an inner surface defining a bore extending to a distal opening at the distal end of the elongate tube;
an outer cannula extending distally from the proximal hub, the outer cannula comprising an outer surface, a proximal end, a distal end, and an inner surface defining a bore extending to a distal opening at the distal end of the outer cannula,
wherein the outer cannula surrounds at least a proximal end region of the elongate tube forming an annular space between the outer surface of the elongate tube and the inner surface of the outer cannula, and wherein the distal end of the outer cannula is located a distance proximal to the distal opening of the elongate tube; and
a lubricious coating on the outer surface of the outer cannula and the outer surface of the elongate tube extending distal to the distal end of the outer cannula, the lubricious coating covering at least a portion of the working length of the elongate needle structure.

2. The device of claim 1, wherein the lubricious coating is a silicone material.

3. The device of claim 2, wherein the silicone material is polydimethylsiloxane applied to the portion of the working length using a solvent carrier.

4. The device of claim 3, wherein the polydimethylsiloxane is at a concentration of 4-7% w/w in the solvent carrier.

5. The device of claim 3, wherein the polydimethylsiloxane has a viscosity in a range of 1,000 cS to about 13,000 cS.

6. The device of claim 1, wherein the at least a portion of the working length covered by the lubricious coating includes a length from a distal-most end of the needle structure to at least about 2 mm from the distal-most end of the needle structure and no greater than about 5 mm from the distal-most end of the needle structure.

7. (canceled)

8. The device of claim 1, wherein the lubricious coating covers at least about 50% of the working length of the elongate needle structure starting at a distal-most end of the needle structure up to about 95% of the working length of the elongate needle structure starting at the distal-most end of the needle structure.

9. The device of claim 1, wherein the proximal hub has a proximal end that is configured to removably couple to a syringe for delivery of a therapeutic from the syringe through the bore of the elongate tube.

10. (canceled)

11. The device of claim 1, wherein the outer cannula has a region that changes in outer diameter towards the distal end of the outer cannula so that an inner diameter of the outer cannula is smaller at the distal end compared to an inner diameter of the outer cannula near the proximal end of the outer cannula.

12. (canceled)

13. The device of claim 1, wherein the outer cannula comprises at least one opening extending through a wall of the outer cannula into the annular space between the outer cannula and the elongate tube.

14. The device of claim 13, wherein the lubricious coating extends over the side wall of the outer cannula at a level of the at least one opening and terminates a distance from the proximal hub.

15. (canceled)

16. The device of claim 1, wherein the outer cannula comprises a plurality of openings extending through a wall of the outer cannula into the annular space.

17. The device of claim 16, wherein the plurality of openings are positioned circumferentially around a longitudinal axis of the outer cannula.

18. The device of claim 16, wherein the plurality of openings are positioned at a plurality of axial locations along a longitudinal axis of the outer cannula.

19. The device of claim 1, wherein the distal end of the elongate tube comprises a sharpened beveled tip.

20.-29. (canceled)

30. A method of lubricating a dual lumen exchange needle, the method comprising:
dip-coating a length of a dual lumen exchange needle into a volume of a silicone material dissolved into a solvent carrier in a concentration of 4-7% w/w, the dual lumen exchange needle comprising:
an inner tube comprising an outer surface, a distal end, and a bore extending to a distal opening at the distal end of the inner tube; and
an outer cannula comprising an outer surface, a distal end, and a bore extending to a distal opening at the distal end of the outer cannula, wherein the distal end of the outer cannula is located a distance proximal to the distal opening of the inner tube, and
wherein the outer cannula surrounds at least a proximal end region of the inner tube forming an annular space between the outer surface of the inner tube and the inner surface of the outer cannula;
applying a positive pressure through the bore of the inner tube while advancing the needle into the first material;
applying a positive pressure through the bore of the outer cannula after withdrawing the needle from the first material; and
forming a coating on the length. (original) The method of claim 30, further comprising evaporating the solvent carrier under infrared light.

32. The method of claim 30, wherein dip-coating comprises a residence time of at least 0 seconds up to about 3 seconds within the material.

33. The method of claim 30, further comprising plasma treating the coating to at least partially cross-link and immobilize silicone to the length.

34.-44. (canceled)

45. A method of refilling an intraocular drug delivery device with an amount of a therapeutic agent, the method comprising:
penetrating a septum of the intraocular drug delivery device with a dual-lumen needle, the drug delivery device at least partially implanted in a vitreous chamber of an eye so the septum is located outside a sclera of the eye and a reservoir of the drug delivery device is located at least partially inside the vitreous chamber,
wherein the dual-lumen needle comprises:
an elongate tube comprising an outer surface, a distal end, and an inner surface defining a bore extending to a distal opening at the distal end of the elongate tube;
an outer cannula comprising an outer surface, a distal end, and an inner surface defining a bore extending to a distal opening at the distal end of the outer cannula,
wherein the outer cannula surrounds at least a proximal end region of the elongate tube forming an annular space between the outer surface of the elongate tube and the inner surface of the outer cannula, and wherein the distal end of the outer cannula is located a distance proximal to the distal opening of the elongate tube; and
a lubricious coating on the outer surface of the outer cannula and the outer surface of the elongate tube extending distal to the distal end of the outer cannula; and
preventing separation of the septum from the intraocular drug delivery device upon the penetrating due to the lubricious coating.

46.-48. (canceled)
